(19) United States
(12) Patent Application Publication
(10) Pub. No.: US 2026/0283771 A1
(43) Pub. Date: Sep. 24, 2026

(54) LOW-COST CAMERA-BASED ESTROUS TRACKING ENABLES TRANSGENESIS IN PEROMYSCUS LEUCOPUS, THE PRIMARY RESERVOIR FOR LYME DISEASE

(71) Applicant: Massachusetts Institute of Technology, Cambridge, MA (US)

(72) Inventors: Joanna Buchthal, Cambridge, MA (US); Emma Chory, Cambridge, MA (US); Christy Dennison, Cambridge, MA (US); Kevin Michael Esvelt, Cambridge, MA (US)

(73) Assignee: Massachusetts Institute of Technology, Cambridge, MA (US)

(21) Appl. No.: 19/474,416

(22) PCT Filed: Apr. 12, 2024
(86) PCT No.: PCT/US24/24403

§ 371 Date: Oct. 10, 2025

Related U.S. Application Data
(60) Provisional application No. 63/495,928, filed on Apr. 13, 2023.
(60) Provisional application No. 63/592,038, filed on Oct. 20, 2023.

(51) Int. Cl. A61D 17/00

(52) U.S. Cl. CPC A61D 17/002

(57) ABSTRACT

Systems and methods described herein provide techniques for identifying a reproductive state of one or more subjects. The systems and methods, in some embodiments, processes data relating to movement activity and is used to identify the reproductive state of the subject. The systems and methods may use one or more machine learning models.

### RELATED APPLICATIONS

This application claims benefit under 35 U.S.C. § 119(e) of U.S. Provisional application Ser. No. 63/495,928, filed Apr. 13, 2023 and U.S. Provisional application Ser. No. 63/592,038, filed Oct. 20, 2023, the disclosure of each of which is incorporated by reference herein in its entirety.

### GOVERNMENT SUPPORT

This invention was made with Government support under Grant No. 1R01AI152209-01 awarded by the National Institutes of Health, TB160101 W81XWH-17-1-0669 awarded by the Department of Defense. The Government has certain rights in the invention.

### FIELD OF THE INVENTION

The invention, in some aspects relates to methods and systems for identifying a reproductive state of a subject.

### BACKGROUND

CRISPR/Cas9 technology has revolutionized the production of animal models by reducing experimental timelines, slashing costs, and streamlining gene editing, leading to a rapid expansion in the number of unique models for the study of human disease and gene function. However, even with the arrival of CRISPR, many wild rodent species, including potential models for aging, cancer, and the study of infectious disease, remain recalcitrant to engineering without effective reproductive technologies required for transgenesis. Due to low pregnancy rates, the lack of external copulatory plugs, and susceptibility to premature termination of pregnancy, wild mice, including those that transmit many human diseases, remain particularly difficult to genetically engineer.

Peromyscus have been put forth as a model organism for human disease, as they appear to more closely resemble humans than laboratory mice by hematological measurements, methylation profiles, disease susceptibility, genetic diversity, and lifespan (which exceeds Mus in captivity by about threefold) [Sun Y, et al., Int. J. Exp Pathol. 2014 October;95(5):342-50; Horvath, S., et al., GeroScience, 44(1), 447-461 (2022); and Havighorst et al., Seminars in Cell & Developmental Biology, 61, 150-155 (2017)]. However, it is widely acknowledged that the use of Peromyscus as a disease model has been impeded by the dearth of genomic tools and technologies available for this species [Havighorst et al., Seminars in Cell & Developmental Biology, 61, 150-155 (2017); Veres et al., Theriogenology, 77 (2012]). Efforts to utilize Peromyscus have been hampered by poor results in previous efforts to generate pseudopregnant mice or engineered fetuses.

For successful transgenesis work and other reproductive technologies, efficient embryo generation and detection are important. As both are well established in Mus, timed pregnancy is routinely employed to generate embryos for transgenesis. Similarly, hormone stimulation has been optimized in Mus to generate large numbers of embryos for experiments, and mating is easily detectable by the presence of copulatory plugs, as mating events are highly correlated with fertilization. Attempts to apply conventional Mus reproductive protocols to other mouse strains, a non-limited example of which is the wild mouse strain Peromyscus, have been markedly unsuccessful.

Without visible copulatory plugs, researchers cannot use plug checking to detect mating, the most commonly employed technique for mating detection in Mus. As a result, prior methods for detecting mating in other mouse strains have included vaginal lavage for estrus and mating detection. However, in strains of mice other than Mus, the estrous cycle has been found to be highly variable using these tools. For example, in Peromyscus the estrous cycle lasting between 2 and 8+ days, despite having an assumed estrous cycle of 4 days (mode=4, median=4.9, average=6) [Dewsbury et al., Journal of Mammalogy, 58(1), 89-92 (1977)]. This variation greatly reduces mating efficiency in a variety of mouse strains. Further, using vaginal lavage to detect sperm is even less predictive of fertilization events, as researchers have routinely found sperm in vaginal smears from nongravid P. leucopus [Dewsbury & Lanier, Physiology & Behavior, 17(6), 921-924 (1976)]. As a result, timed mating with the aforementioned tools has been reported to be highly inefficient in mouse strains, including for example P. leucopus, with a published pregnancy rate of 20% in females mated with males to satiety [Dewsbury & Lanier, Physiology & Behavior, 17(6), 921-924 (1976)].

In efforts to increase pregnancy rates (and thus embryo yields) in non-Mus subjects, previous methods have combined superovulation, timed mating, and artificial insemination, but still only produced a fraction of the embryos that are typically harvested by superovulation alone in Mus [Veres et al., Theriogenology, 77 (2012)]. Finally, because mating events in strains can regularly occur without giving rise to pregnancy, more recent advances such as mating detection with cameras are also likely to be unsuccessful in certain strains, despite success in other species [(Doroba et al., Anatomical Record, 293(8), 1325-1332 (2010); Lo et al., Proceedings: APSIPA ASC 2009: Asia-Pacific Signal and Information Processing Association, 2009 Annual Summit and Conference, 668-671 (2009)]. Thus, without reliable reproductive protocols for efficiently generating fertilized embryos, genetic engineering, and reproductive efficiency in many strains of mice and in other mammals cannot advance. The lack of ability to identify reproductive states in strains of mice, rodents, and in other species negatively impacts research and prevents implementation of reproductive advances in mammalian subjects.

### BRIEF DESCRIPTION OF THE DRAWINGS

FIG. 1A-M provides schematic diagrams, graphs, and heat maps. FIG. 1A provides a schematic diagram of iGONAD: Mice are mated and then checked for signs of pregnancy. CRISPR/Cas9 is injected into the oviduct of a pregnant mouse, and then the oviduct is electroporated. FIG. 1B is a chart of iGONAD statistics including all procedures performed in Peromyscus. FIG. 1C is a graph showing the Mus musculus estrous cycle. Ovulation occurs every 4 to 5 days. FIG. 1D is a schematic diagram of mouse telemetry system: G2 E-Mitter is implanted in the abdominal cavity in order to detect core body temperature and activity data over time. FIG. 1E is a schematic diagram illustrating the G2 E-Mitter transmitting data at 1 min intervals to a receiver, which is positioned under an animal's cage and communicates with the Vital View Telemetry Software. FIG. 1F is a map showing core body temperature data from two individual female Peromyscus with sequential days stacked from top to bottom. Prolonged peaks in core body temperature (estrus) appear in 4-day intervals. X axis: 0=8 pm when lights turn off in the animal facility. Y axis: 0=first day after surgery. FIG. 1G is a map of mean core body temperature for 11 female Peromyscus and shows no variability in cycle length among cycling mice. FIG. 1H is a graph illustrating estrous cycling rate among all Peromyscus with implanted G2 E-Mitters compared to previously published cycling rates. FIG. 1I is a schematic diagram of two different Peromyscus mating schemes. Top: Mating when mice are in estrus leads to the suspension of the estrous cycle. Bottom: When mice are mated in diestrus, the estrous cycle continues unabated. FIG. 1J is a heat map of mean core body temperature for 15 female Peromyscus mated in estrus. Sequential days are stacked from top to bottom from time of mating with stud males. Estrous cycle disruption is clearly visible after mating, indicating a high rate of pregnancy and pregnancy-like events. FIG. 1K is a heat map of mean core body temperature for 13 female Peromyscus mated in diestrus. All mice continued to cycle after mating. FIG. 1L shows a graph illustrating that when compared, morning core body temperature for pregnant mice is distinct from non-pregnant mice, which show no disruption of morning temperature cycling when mated in diestrus. X axis: average core body temperature in the morning (after lights turn on). Y axis: 0=mating in diestrus or estrus. FIG. 1M is a graph showing efficiency of timed pregnancy using core body temperature for estrous tracking compared with previously reported timed pregnancy rates in Peromyscus using vaginal lavage and cytology.

FIG. 2A-L provides schematic diagrams, graphs, and maps from a study of tracking Peromyscus activity. FIG. 2A is a schematic diagram of camera system for tracking Peromyscus activity. Mouse movement was detected with a Raspberry Pi NoIR Camera V2 module attached to a Raspberry Pi Zero 2 W computer with WiFi. FIG. 2B is a photographic image showing Raspberry Pi units that were attached to acrylic stands that were positioned behind every cage on the cage rack. FIG. 2C is a schematic diagram of data export and analysis. Images captured by Raspberry Pi cameras were processed on the Raspberry Pi units. The activity data was uploaded to the cloud database and then downloaded to the server and displayed on the website. FIG. 2D is a diagram of web interface. Active Raspberry Pi units were listed on the “All Cameras” page, along with their status and IP address, for equipment monitoring and troubleshooting. All mice were listed on the “All Mice” page with a click-through to individual mice, their activity graphs and latest photo to monitor for obstructions. FIG. 2E are maps showing comparison of mouse estrous tracking systems for a single mouse using either core body temperature or activity from the mouse telemetry system, or activity from a Raspberry Pi unit. FIG. 2F are maps showing comparison of all three estrous tracking systems on a different mouse, with a near-IR LED attached to the Raspberry Pi unit for night-time tracking. FIG. 2G is a graph of cycling status of the first 500 mice placed on camera. FIG. 2H is a graph showing distribution of ages at which estrus-cycle-linked activity was first detected. FIG. 2I is a map showing results of an example of early estrous onset. The first estrus behavior can be distinguished from non-estrous activity when no discernible 4-day trends are present. X axis: 0=8 pm when lights turn off in the animal facility. Y axis: 0=first estrus day. FIG. 2J is a map showing results of a rare example of Peromyscus with 5-day estrous cycle. FIG. 2K is a map showing results of an example of mouse with shifted circadian rhythm. FIG. 2L is a map showing results of an example of mouse with shifted circadian rhythm and visible estrous cycling behavior.

FIG. 3A-G provides schematic diagrams and graphs illustrating results. FIG. 3A is a schematic diagram of Mus and Peromyscus hormone priming protocols. FIG. 3B is a graph showing pooled results from superovulation experiments, in which mice were hormone primed with 72 hours between PMSG and HCG. The average embryo yield per mouse was highly variable between experiments. FIG. 3C is a graph illustrating results in which cells harvested from individual mice following the 72 hour superovulation protocol, demonstrated low numbers of both 1 and 2 cell embryos, with outliers. FIG. 3D is a graph showing a comparison of fertilization rate following either the 72 hr or 48 hr superovulation protocols; both produced equally low rates of fertilized cells. FIG. 3E is a graph of cells harvested from individual mice after timed mating without superovulation. Timed mating resulted in more fertilized embryos per mouse and less variability in cell yield. FIG. 3F is a graph showing fraction of cell types harvested from superovulated or timed-mated mice. FIG. 3G is a graph showing cells harvested from timed-mated mice segmented by age. Younger mice produced higher 2-cell embryo yields than older mice, which were more likely to produce unfertilized eggs.

FIG. 4A-I provides schematic diagrams, photomicrographic images, graphs and maps illustrating results. FIG. 4A is a schematic diagram of mating scheme for pseudopregnancy in Peromyscus. Mating in estrus with a vasectomized male is expected to produce pseudopregnant females with suspended cycles. FIG. 4B is a heat map of mean core body temperature for 6 mice with implanted sensors that had been mated in estrus with vasectomized males. Estrous cycle suspension is visible after mating, indicating a high rate of pseudopregnancy. X axis: 0=8 pm when lights turn off in the animal facility. Y axis: 0=mating day. FIG. 4C (left) is a mean activity plot for 53 camera-tracked mice mated with vas males in estrus. 85% of mice exhibited suspended estrous cycling. FIG. 4C (right) shows the number of mice per time point. FIG. 4D provides photographs of early gestational time points, including zygote development, from timed-mated mice. The pronuclei only became visible 18 hours after timed matings were set up. FIG. 4E is a table showing distribution of cells harvested on gestational days e2.5 through e5.5 from timed-mated mice. FIG. 4F is a diagram of Mus and Peromyscus leucopus early gestational timelines. Peromyscus embryos stay in the 2-cell stage longer than Mus embryos and require considerably more time to reach the blastocyst stage. FIG. 4G is a graph showing an in vitro developmental timeline of Peromyscus embryos harvested at the 2-cell stage and cultured in KSOM until blastocysts formed. FIG. 4H is a graph of results of cells recovered from individual uterine horns from timed-mated mice on gestation day e1.5. FIG. 4I is an image showing pronuclear injection statistics for the 6 recipients that each received 4 embryos injected with Cas9, Rosa26 guide and the EGFP targeting construct.

FIG. 5A-F provides schematic diagrams, graphs, and heat maps of results. FIG. 5A is a schematic diagram of a phylogenetic tree illustrating the genetic distance between the genus Mus (from the Muridae family) and the genus Peromyscus and Mesocricetus or hamsters (from the Cricetidae family). Adapted from Bedford and Hoekstra [Bedford et al., elife, 4 (2015)]. FIG. 5B is a Mus activity heatmap generated from image difference analysis showcasing a 5-day cycling pattern (indicated by white arrows) 5 to 8 hours after lights off. FIG. 5C are graphs of Mus daily cumulative activity plot with a 5-day color scheme (top) and its autocorrelation (bottom), post-sectioning and background removal; autocorrelation shown with a 95% confidence interval. FIG. 5D is a Peromyscus activity heatmap (left), daily cumulative activity plot with a 4-day color scheme (top right) and autocorrelation with a 95% confidence interval (bottom right). All derived from image difference analysis without sectioning and background removal. FIG. 5E is a Mus activity heatmap (left), daily cumulative activity plot with a 4-day color scheme (top right), and its autocorrelation (bottom right) post sectioning and background subtraction. FIG. 5F is a hamster activity heatmap detected by thermal camera (left), corresponding daily cumulative activity plot with a 4-day color scheme (top right) and its autocorrelation post sectioning and background subtraction with a 95% confidence interval (bottom right).

FIG. 6A-J provides schematic diagrams, graphs, and plots showing results relating to camera-based activity tracking for monitoring female reproductive aging. FIG. 6A is a schematic diagram of vaginal swabbing timeline. Groups of 4 Peromyscus were swabbed every 6 hours from midnight on the night of ovulation to collect samples from the entire estrous cycle. FIG. 6B is a plot of vaginal cells collected from individual mice organized by their collection time relative to ovulation. For each mouse, each cell type (cornified, leucocyte or nucleated) was counted and averaged across 3 images. FIG. 6C is a schematic diagram of the cell types found in vaginal smears and their relative abundances across the 4 stages of the estrous cycle in mice, rats (adapted from Cora et al., 2015), and Peromyscus. FIG. 6D shows a confusion matrix computed using validation set. Days from ovulation assumes ovulation happens at midnight on day one. FIG. 6E shows multi-class ROC curves for each day after ovulation computed using validation set. FIG. 6F shows gradient-weighted class activation maps overlaid on images from day 1 post ovulation (left) and day 3 post ovulation (right). FIG. 6G provides count of images classified for each test mouse by day post ovulation. FIG. 6H shows a table of mice chosen for histological analysis, including mouse ID, age when analyzed, mating status and histological classification. FIG. 6I shows a photograph of H&E stained ovary, uterus and vagina sections from all three cycling mice (IDs 1122, 1047, 1118) on “estrus” day. Organs show histological features of estrus, including large antral follicles (top left, top middle), basophilic corpus lutea (top right, darker arrows), apoptotic cells with tall columnar endometrium (middle row, lighter arrows) and a vaginal cornified layer with sloughed cells and debris in the lumen (bottom row). FIG. 6J provides images of H&E stained ovary, uterus and vagina sections from all three non-cycling mice (IDs 1117, 1116, 1048). Organs show signs of aging, including evidence of persistent estrus (mouse ID: 1117) with hyperplastic endometrial epithelium and early endometrial hyperplasia (middle left) and vaginal epithelium cornification (bottom left); persistent diestrus (mouse ID: 1116) with low, cuboidal, inactive endometrial epithelium (middle center) and low, but mucified vaginal epithelium (bottom center); mouse nearing anestrus (mouse ID: 1048) with an atrophic ovary (top right), endometrial cystic hyperplasia/pyometra (middle right) and cornified vaginal epithelium with intraluminal cell debris and hemorrhage (bottom right).

FIG. 7 provides graph of timed mating results. Gestational length and litter size for 11 mice that produced pups after timed mating. While the date of birth was noted for all 11 litters, pups were only counted in 8 of the 11 litters, as litters were cannibalized.

FIG. 8A-C provides heat map results of sensor activity data. FIG. 8A is a heat map of the ratio of core body temperature and activity and shows that temperature readouts are highly correlated with corresponding activity data. FIG. 8B provides activity data from three individual female Peromyscus. Prolonged extensions of activity (estrus) coincide with elevated temperatures. X axis: 0=8 pm when lights turn off in the animal facility. Y axis: 0=first day after surgery. FIG. 8C shows mean activity for 11 female Peromyscus shows activity consistently aligns with temperature trends. X axis: 0=8 pm when lights turn off in the animal facility. Y axis: 0=first estrus day after surgery.

FIG. 9 provides a graph that compares mating outcomes. Morning core body temperature for pregnant and pseudopregnant Peromyscus is similar, though the suspension of the estrous cycle is truncated in pseudopregnant mice. No disruption of morning temperature cycling is observed when Peromyscus are mated in diestrus.

FIG. 10 provides image difference analysis. The original camera tracking system applies pixel-wise subtraction on two successive images and then sums the absolute values of the differences to produce an activity metric. However, if two successive images were similar in color when under IR light (like image 1 and image 2), the resulting image difference mask can fail to capture animal activity.

FIG. 11 is a graph of results indicating Mus musculus with shifting high activity day. Daily cumulative activity plot for Mus musculus with shifting high activity day. Arrows point to high activity days, which appear to shift from one 4-day cycle to another 4-day cycle. X axis: daily cumulative activity 5 to 8 hours after lights off. Y axis: sequential days from left to right.

FIG. 12 is a schematic diagram of an example of Peromyscus transgenesis process.

### SUMMARY OF CERTAIN ASPECTS OF THE INVENTION

According to an aspect of the invention, a method of identifying a reproductive state in a female mammalian test subject is provided, the method including: (a) measuring a level of movement activity of the female test subject in a first time period, wherein the movement activity is measured in the absence of a mating activity of the female test subject; (b) comparing the level of movement activity of the female test subject measured in the first time period with a control level of movement activity; and (c) determining a difference in the measured level of movement activity of the female test subject in the first time period compared to the control level of movement activity; wherein the presence of the determined difference assists in identifying the reproductive state in the female test subject. In some embodiments, the method also includes simultaneously measuring the level of movement activity in a plurality of test subjects. In certain embodiments, the plurality of test subjects is in a single cage or in a plurality of cages. In some embodiments, the measuring includes one or more of: detecting the movement activity with one or a plurality of sensors; visually detecting the movement activity; and imaging the movement activity with one or a plurality of cameras, optionally wherein one or more of the one or plurality of cameras is a thermal-imaging camera. In some embodiments, the plurality of sensors or the plurality of cameras is at least 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 400, 500, or more sensors or cameras, respectively. In certain embodiments, the sensor includes one or more of an RFID sensor, a vibration detecting sensor, a noise detecting sensor, and a pressure detecting sensor. In certain embodiments, the sensor or plurality of sensors are located at one or more of: inside the subject; inside the cage containing the subject; and outside the cage containing the subject. In some embodiments, the identified reproductive state includes at least one of proestrus, estrus, metestrus, diestrus, anestrus, ovulation, pseudopregnancy, and pregnancy. In some embodiments, the method also includes initiating a pregnancy in the test subject when the identified reproductive state of the test subject is estrus, optionally is ovulation. In some embodiments, the method also includes initiating a pseudopregnancy in the test subject when the identified state of the test subject is estrus, optionally is ovulation. In certain embodiments, the method also includes not initiating a pregnancy or pseudopregnancy in the test subject when the identified state of the test subject is not estrus. In certain embodiments, the method also includes not initiating a pregnancy or pseudopregnancy in the test subject when the identified state of the test subject is not ovulation. In some embodiments, the method also includes initiating an environmental or physical change for the test subject based on the reproductive state of the subject. In some embodiments, the reproductive state of the subject is estrus, optionally ovulation, and the physical change includes one or more of a surgery on the subject and artificial insemination of the subject. In certain embodiments, the reproductive state of the subject is not estrus and one or more of the environmental changes and the physical change initiated for the test subject induces estrus, optionally induces ovulation. In certain embodiments, determining the difference in the measured level of movement activity of the test subject in the first time period compared to the control level of movement activity includes one or more of subtracting pixels and training a machine learning (ML) model. In some embodiments, the training of the ML model includes training the ML model to predict the level of movement activity of the one or plurality of subjects. In certain embodiments, the training of the ML model includes training the ML model with one or more of a plurality of individual frames as inputs. In certain embodiments, the training of the ML model includes training the ML model with one or a plurality of a portion of a frame as an input. In some embodiments, the absence of an increase in the measured level of movement activities of the test subject compared to the control level of movement activities identifies the reproductive state of the test subject as including one of: proestrus, estrus, metestrus, diestrus, anestrus, ovulation, pseudopregnancy, and pregnancy. In some embodiments, the presence of an increase in the measured level of movement activities of the test subject compared to the control level of movement activities identifies the reproductive state of the test subject as estrus. In certain embodiments, the presence of an increase in the measured level of movement activities of the test subject compared to the control level of movement activities identifies the reproductive state of the test subject as ovulation. In certain embodiments, the control movement activities are not estrus-cycle-linked activities and the presence of an increase in the level of the measured movement activities of the test subject compared to the control level of movement activities identifies the reproductive state of the test subject as estrus. In certain embodiments, the control movement activities are estrus-cycle-linked activities and the presence of a decrease in the level of the measured movement activities of the test subject compared to the control level of movement activities identifies the reproductive state of the test subject as not an estrus state. In some embodiments, the control movement activity includes movement activity of the test subject in a second time period. In some embodiments, the control movement activity includes movement activity of the test subject in a time period when the test subject is not in estrus. In certain embodiments, the control movement activity includes movement activity of the test subject in a time period when the reproductive state of the test subject includes at least one of proestrus, metestrus, diestrus, anestrus, ovulation, pseudopregnancy, and pregnancy. In certain embodiments, the control movement activity includes movement activity measured in a one or a plurality of different subjects of the same species as the test subject. In some embodiments, the one or plurality of the different subjects are not in estrus. In some embodiments, the reproductive state of the one or plurality of the different subjects is proestrus, metestrus, diestrus, ovulation, pseudopregnancy, or pregnancy. In certain embodiments, the measurement includes detecting the movement activity. In certain embodiments, the detecting includes imaging the test subject's movement activity with a camera or other visual recording device. In some embodiments, the measuring of the movement activity of the test subject includes obtaining a plurality of images of the test subject at preselected time intervals and comparing two or more of the obtained plurality of images to each other as a measure of movement activity of the test subject. In some embodiments, the preselected time intervals are from 0.5-1.5 seconds in length, from 0.5 to 2.5 seconds in length, from 1 to 3 seconds in length, from 1 to 5 seconds in length, from 1 to 10 seconds in length, or from 1 to 20 seconds in length, from 1 to 60 seconds in length, from 20 to 60 seconds in length, from 1 to 2 minutes in length, from 1 to 5 minutes in length, from 1 to 10 minutes in length, from 5 to 10 minutes in length, and from 5 to 30 minutes in length, including all time lengths in each range. In some embodiments, the preselected time intervals are at least 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100, 110, 120, 130, 140, 150, 160, 170, 180, 190, 200, 300, 400, 500, 600, 700, 800, 900, or 1000 seconds in length, including each time length between each recited integer. In certain embodiments, the method also includes performing pixel-level analysis of the two or more obtained images to detect the presence or absence of differences between the two or more images, wherein the detected differences identify movement activity by the test subject. In certain embodiments, the method also includes identifying a level of movement activity of the test subject in the first time period and in a second time period. In some embodiments, identifying the level of movement activity of the test subject in the first time period includes comparing the pixel-level analysis of two or more of the images obtained in the first time period. In some embodiments, the method also includes comparing the identified level of movement activity in the first time period to a level of activity identified in the second time period. In certain embodiments, the second time period either precedes or is subsequent to the first time period. In certain embodiments, the length of the first time period is up to 24-hours. In some embodiments, the length of the second time period is up to 24-hours. In some embodiments, the test subject is a rodent, optionally a mouse or a hamster. In some embodiments, the rodent is a wild rodent, optionally a wild mouse or a wild hamster. In certain embodiments, the rodent is a genetically engineered rodent. In certain embodiments, the rodent is a spiny mouse or a naked mole rat. In certain embodiments, the test subject is a genetically engineered test subject. In some embodiments, the test subject is a non-rodent mammal. In certain embodiments, the non-rodent mammal is a tree shrew or a marmoset. In some embodiments, determining an increase in the test subject's measured movement activity in a 24-hour period compared to one, two, three or more consecutive 24-hour periods assists in identifying the stage of the estrous cycle of the test subject. In some embodiments, the method also includes administering a candidate therapeutic agent to the test subject, at least one or more of: before, during, and after the measuring. In some embodiments, the method also includes monitoring the subject and identifying one or more of a presence, an absence, and a level of an effect of the administered candidate therapeutic agent on the test subject. In certain embodiments, the female subject is not a horse or a cow. In some embodiments the test subject is a geriatric/elderly test subject. In some embodiments, the method also includes assessing an age-related change in a reproductive state of the subject.

According to another aspect of the invention, a computer-implemented method of identifying a reproductive state in a female mammalian test subject is provided, the method including: (a) receiving imaging data representing a plurality of images capturing movement activity of the female test subject, wherein the movement activity images are captured in the absence of a mating activity of the female test subject; (b) measuring, using the imaging data, a level of movement activity of the female test subject in a first time period; (c) comparing the level of movement activity measured in the first time period with a control level of movement activity; and (d) determining a difference in the measured level of movement activity of the female test subject in the first time period compared to the control level of movement activity; wherein the presence of the determined difference assists in identifying the reproductive state in the female test subject. In some embodiments, the method also includes processing the imaging data using a trained ML model to determine the level of movement activity. In some embodiments, the imaging data represents camera images capturing movements of more than one subject. In certain embodiments, the images are camera images, optionally thermal-imaging camera images. In certain embodiments, the female test subject is a rodent, optionally a mouse or a hamster. In some embodiments, the rodent is a wild rodent, optionally a wild mouse or a wild hamster. In some embodiments, the rodent is a genetically engineered rodent. In certain embodiments, the rodent is a spiny mouse or a naked mole rat. In certain embodiments, the female test subject is a genetically engineered test subject. In some embodiments, the test subject is a non-rodent mammal. In certain embodiments, the non-rodent mammal is a tree shrew or a marmoset. In some embodiments, the female subject is not a horse or a cow. In some embodiments the test subject is a geriatric/elderly test subject. In some embodiments, the method also includes assessing an age-related change in a reproductive state of the subject.

### DETAILED DESCRIPTION

Methods and systems of the invention permit low-cost activity-based ovulation tracking for the efficient and high throughput generation of pregnant and pseudopregnant subjects. The present disclosure relates to methods for identifying a reproductive state in a subject. In some embodiments, a method of the invention comprises a computer-implemented method. Certain embodiments of methods and systems of the invention can be used with subjects such as, but not limited to rodents, to unlock genome engineering in wild species of organisms. Leveraging this technology, use of embodiments of systems and methods of the invention have resulted in efficient generation of engineered Peromyscus leucopus, the primary reservoir for Lyme disease-causing bacteria. Genetic engineering of this genus, which has considerable research value due to its role in disease transmission and longevity, marks the dawn of Peromyscus as a genetic model, and the advent of more facile genetic engineering of organisms such as, but not limited to, rodents with broad value in ecology, human health, and disease. Methods and systems of the invention can be used for test subjects that are mice or other species, a non-limiting example of which is Syrian hamsters, Mesocricetus auratus. Results of certain studies described herein demonstrate successful application activity-based ovulation tracking using embodiments of methods and/or systems of the invention for the efficient and high throughput generation of pregnant and pseudopregnant hamsters.

Although plants and animals have been engineered for centuries through selective breeding, scientists only recently gained the tools to make deliberate genome modifications [Sharan, S. K., et al., Nat. Protoc. 4, 206-223 (2009)]. The advent of transgenic plant and animal technology has enabled major advances in research and biotechnology with striking examples in pharmaceutical production, foreign organ transplantation and agriculture. In particular, genetically engineered mouse models have become indispensable tools for studying human disease, development, and gene function. Embodiments of methods and systems of the invention can be used to readily generate animal models with broad applications.

Methods and systems described herein can be used to enable use of Peromyscus as a tractable model organism. Methods and systems provided herein present optimized reproductive protocols for the generation of timed pregnant and pseudopregnant Peromyscus, which rival the efficiency of timed matings in laboratory mice. Use of embodiments of methods and systems of the invention, have demonstrated that efficient embryo transfer is achievable in P. leucopus, thereby unlocking the use of the most powerful transgenesis techniques, namely zygote and stem cell-based technologies, in this species. Finally, certain embodiments of methods and systems of the invention can be used in efficient Peromyscus genome engineering with CRISPR/Cas9 or other gene-editing systems, paving the way for the development of Peromyscus as homologous, isomorphic and predictive disease models, as well as their use for ecological engineering projects, such as Mice Against Ticks, a community-guided effort to engineer Lyme-resistant Peromyscus [Buchthal et al., Philosophical Transactions of the Royal Society of London. Series B, Biological Sciences, 374(1772), 20180105 (2019)].

The invention, in part, includes use of camera-based activity tracking to significantly increase the accuracy of Peromyscus vaginal cytology and generate training data for a machine learning model to further improve cytology analysis for resource-constrained labs. In addition, certain embodiments of methods of the invention disclosed herein include camera-based activity tracking that successfully distinguishes reproductively healthy, old Peromyscus from those showing signs of reproductive aging, thereby providing a new, non-invasive tool for investigating the mechanisms that govern female reproductive health, while advancing Peromyscus as an aging model.

In some embodiments of methods and systems of the invention are used in hamsters. Methods and systems provided herein present optimized reproductive protocols for the generation of timed pregnant and pseudopregnant hamsters. Use of embodiments of methods and systems of the invention, have demonstrated that efficient embryo transfer is achievable in hamsters, thereby unlocking the use of the most powerful transgenesis techniques, namely zygote and stem cell-based technologies, in this species. Finally, certain embodiments of methods and systems of the invention can be used in efficient hamster genome engineering with CRISPR/Cas9 or other gene-editing systems, paving the way for the development of hamsters as homologous, isomorphic, and predictive disease models, as well as their use for ecological engineering projects.

Certain embodiments of methods and systems of the invention, include combining high-efficiency estrus detection with CRISPR/Cas9 genome engineering, which can permit the generation of wild P. leucopus that heritability express anti-Lyme antibodies derived from locally sourced mice [Schaible et al., Proceedings of the National Academy of Sciences of the United States of America, 87(10), 3768-3772 (1990)]. Consequently, Peromyscus engineered using embodiments of methods and systems of the invention can serve as proof of concept for modifying rodents to intervene against a variety of rodent-maintained zoonoses, ranging from Lassa fever to hantavirus pulmonary syndrome. For example, though not intended to be limiting, embodiments of methods and systems of the invention may be used to prepare engineered P. leucopus that are heritably resistant to B. burgdorferi, the etiological agent of Lyme disease [Telford et al., Lyme Disease and Relapsing Fever Spirochetes: Genomics, Molecular Biology, Host Interactions and Disease Pathogenesis (2021)]. Such resistant engineered P. leucopus may be released into the wild wherein their interbreeding with the wild population of P. leucopus can result in reduced transmission of Lyme disease by an increase in the level of inherited resistance to B. burgdorferi in the wild population of P. leucopus. Thus, certain embodiments of methods and systems of the invention may be used to break the disease transmission cycles between wild mice and ticks, and reduce disease prevalence in hard-hit areas like Nantucket and Martha's Vineyard.

In addition, methods and systems of the invention may be used to generate new rodent models for use in identifying and treating underlying mechanisms that influence characteristics such as, but not limited to longevity, neurogenesis, social behavior patterns, degeneration, and susceptibility to disease.

With over 56 recognized species, the Peromyscus genus is celebrated for its extensive diversity, and embodiments of methods and systems of the invention can be used to unlock genome engineering for all Peromyscus species. In addition, embodiments of methods and systems of the invention can also be used for genome engineering and to prepare model organism in other species, for example, though not limited to other mice, rats, hamsters, rodents, non-rodent mammals, etc., which can enable the modeling of diseases, such as but not limited to human diseases and other mammalian diseases. Collectively, methods and systems of the invention provide tools capable of producing high efficiency timed pregnancy and pseudopregnancy and represent a transformative opportunity for transgenesis in mammals and broadens the use of genetic engineering technologies in recalcitrant and other species.

Methods and systems described herein permit generation of highly efficient pregnancy and pseudopregnancy events in species of mice and other organisms. A non-limiting example of an organism to which methods of the system can be successfully applied is the mouse strain, Peromyscus. As shown herein, methods and systems of the invention may be used to enable genetic engineering in mammals, a non-limiting example of which is the wild rodent species Peromyscus. Certain embodiments of methods and systems of the invention are illustrated herein as applied to Peromyscus leucopus. It will be understood that Peromyscus leucopus is a non-limiting example of a species to which methods of the invention can be applied. In another non-limiting example, certain embodiments of methods and systems of the invention are illustrated herein as applied to hamsters.

Peromyscus leucopus, is the primary reservoir of Borrelia burgdorferi, and engineering advances in this species may be used to identify new ecological tools for combating Lyme disease. Studies provided herein illustrate work performed in the development of a broadly enabling solution for improving mating efficiency, and surprisingly, it was determined that animal movement patterns were indicative of estrous cycle stage, thereby enabling the detection of ovulation (rather than simply mating events) via low-cost camera-tracking. With reliable estrus tracking, it has now been demonstrated that the generation of pregnant and pseudopregnant Peromyscus is in fact achievable with efficiencies nearing Mus, thereby enabling the improved efficiency of all transgenesis techniques in Peromyscus and other strains and species of mammals. Studies and information presented herein, based at least in part on embodiments of reproductive detection methods and systems of the invention, provide evidence of methods of efficient generation of engineered organisms, such as but not limited to Peromyscus, an achievement that marks an expansion of available engineered mammalian animal models. Methods and systems of the invention may be used to generate engineered organisms and may be used to enhance reproductive strategies for mammalian subjects.

In some embodiments of methods and systems of the invention may be used in animal husbandry practices, for example, to streamline animal husbandry practices. As a non-limiting example, an animal facility may utilize methods of the invention to mate mice more efficiently without having to physically check estrous states, a process that can be labor-intensive. In addition, embodiments of methods of the invention can be used to scale animal colonies more effectively and forecast more accurately based on the availability of cycling females. In some embodiments, methods of the invention may be used to assist in breeding difficult-to-breed strains of Mus or other mammals.

Some embodiments of methods of the invention may be used to facilitate embryology applications. As a non-limiting example, methods of the invention can be used to facilitate the generation of embryonic stem cells in some animals. Certain embodiments of methods of the invention may be used to enable the capture of one-cell and two-cell embryos for microinjection and in vitro culture, including ESC derivation from blasts. Blastocysts from timed matings can be used for blast injection and interspecies chimeras, among other applications.

### Methods and Systems

In some embodiments of methods and systems of the invention, a subject may be monitored for its reproductive state. The invention includes, in part, methods and systems that can be used to identify a reproductive state in a mammalian test subject or in a plurality of test subjects. As used herein, the term “plurality” means more than one. For example, in some embodiments a plurality is 2, 3, 4, 5, 6, 7, 8, 9, 10, 20, 30, 40, 50, 60, 70, 100, 200, 500, or more, including all integers between stated numbers. In some embodiments, methods of the invention include: (a) measuring a level of movement activity of a test subject in a first time period; (b) comparing the level of movement activity measured in the first time period with a control level of movement activity; and (c) determining a difference in the measured level of movement activity of the test subject in the first time period compared to the control level of movement activity; wherein the presence of the determined difference assists in identifying the reproductive state in the test subject. In some embodiments of methods and systems of the invention, measuring a level of movement activity of a test subject is done in the absence of a mating activity of the test subject. As used herein in reference to measuring estrus-cycle-linked activity in a female subject, the term “mating activity” means mounting or attempted mounting of the female subject by another subject and/or copulation or attempted copulation by another subject, for example by a male subject. Demonstrations of measuring female subject estrus-cycle-linked activity in the absence of mating activity are provided in figures and in the Examples section herein. As a non-limiting example, in some embodiments, movement activity of a female test subject is measured when the female test subject is not in the presence of a male subject.

As used herein in reference to measuring and comparing levels of movement activity the term “difference” means a detectable difference between two or more levels of movement. In some embodiments, the difference is a statistically significant difference. In certain embodiments, a difference need not be a statistically significant difference and any detectable difference two or more levels is sufficient to indicate a difference.

It will be understood that methods and systems of the invention can be used to identify a estrus-cycle-linked activity and corresponding reproductive state in one subject or in a plurality of subjects. In some embodiments, methods of the invention include simultaneously measuring a level of movement activity more than one subject, for example, in a plurality of test subjects. In certain embodiments of methods and systems of the invention, the one or plurality of test subjects are caged subjects. In embodiments of methods and systems in which the reproductive states of subjects in a plurality of test subjects are measured, all or a portion of the plurality of test subjects may be contained in a single cage or container. In certain embodiments, a plurality of test subjects may be contained in a plurality of cages or containers with one or more subjects in the plurality of cages or containers.

Some embodiments of methods and systems of the invention include computer-implemented methods and systems for identifying a reproductive state in a mammalian test subject. The methods and systems may include (a) receiving imaging data representing a plurality of camera images capturing movement activity of the test subject; (b) measuring, using the imaging data, a level of movement activity of the test subject in a first time period; (c) comparing the level of movement activity measured in the first time period with a control level of movement activity; and (d) determining a difference in the measured level of movement activity of the test subject in the first time period compared to the control level of movement activity, wherein the presence of the determined difference assists in identifying the reproductive state in the test subject. In some embodiments, the computer-implemented methods and systems also include processing the imaging data using a trained machine learning (ML) model to determine the level of movement activity. In certain embodiments of a computer-implemented method or system of the invention, the imaging data represents camera images capturing movements of more than one subject. In some embodiments of methods of the invention, the camera images are thermal images obtained with a thermal-imaging camera.

### Measurement

Embodiments of methods and systems of the invention include measuring movement activity of a subject. In some embodiments, the measuring comprises detecting one or more of overall body movement by the subject, locomotive movement of the subject, and body movement in the absence of locomotive movement. As used herein the term “locomotive movement” is used in reference to a change in physical location of a subject. For example, though not intended to be limiting, a locomotive movement may be walking or running of a subject from an initial physical location. For example, in some instances, measuring a locomotive movement of a subject may begin when the subject moves from an initial physical location, and continue as the subject moves to one or more different physical locations. The measurement of the subject's locomotive movement may start at the time the subject leaves an initial physical location and end either when the subject stops at a position that is not the initial physical location or the when the subject returns to and stops at the initial physical location. As used herein the term “body movement” used in reference to measured movement activity of a subject, means changes in a position of one or more portions of the subject's body that do not result in the subject occupying a different physical location due to the movement. Non-limiting examples of body movement are twitching, grooming, rearing, shaking, whisker movement, sniffing, and head movement. Additional types of body movements can also be measured using methods and systems of the invention.

Embodiments of methods and systems of the invention include measuring movement activity of one or a plurality of subjects. In some embodiments, measuring may include absolute measurements and/or relative measurements of a subject's movement activity. For example, one can detect numbers and/or types of movement activity of a subject and/or can detect changes in a level of movement activity of a subject.

### Measuring Means

In certain embodiments of methods and systems of the invention, measuring movement activity of one or a plurality of subjects includes movement detection. Various types of detection methods and devices can be used to obtain movement information that is used to measure movement activity in methods and systems of the invention. Non-limiting examples of detection methods and devices that may be used in certain embodiments of methods and systems of the invention are sensors that detect movement activity; visual detection of movement activity; and imaging movement activity using one or a plurality of cameras. In some embodiments of methods and systems of the invention, a camera is a thermal imaging camera. In some embodiments of methods and systems of the invention, detecting movement activity with a plurality of sensors or a plurality of cameras means movement activity of one or more subjects is detected with at least 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 400, 500, or more sensors or cameras, respectively.

It will be understood that a variety of sensors are suitable for use to detect movement activity in a method or system of the invention. Non-limiting examples of types of sensors that may be used are one or more RFID sensor, one or more vibration-detecting sensor, one or more noise-detecting sensor, and one or more pressure-detecting sensor. In some embodiments of methods and systems of the invention, one or a plurality of one type of sensor is used and in certain embodiments of methods and systems of the invention two or more different types of sensors are used to detect movement activity of one or a plurality of subjects.

The positioning of one or a plurality of sensors in embodiments of methods and systems of the invention can be located at positions suitable for detecting movement activity of the one or plurality of subjects. Non-limiting examples of where sensors may be located are at one or more of: inside the subject; inside a cage containing the subject; outside a cage containing the subject; and at a location know to be frequented by the subject.

In some embodiments of methods and systems of the invention, measuring movement activity includes detecting the movement activity of a test subject using a camera or a recording device, a non-limiting example of which is a visual recording device. In some embodiments, one or a plurality of cameras used in a method or system of the invention is a thermal imaging camera. In some embodiments, measuring movement activity of a test subject or a plurality of test subjects includes obtaining a plurality of images of the test subject or plurality of test subjects at one or more preselected time intervals and comparing two or more of the obtained plurality of images to each other as a measure of movement activity of the test subject or plurality of test subjects, respectively. In some embodiments of methods and systems of the invention, the images are thermal images. The timing and length of preselected time intervals can range from 0.5-1.5 seconds in length, from 0.5 to 2.5 seconds in length, from 1 to 3 seconds in length, from 1 to 5 seconds in length, from 1 to 10 seconds in length, or from 1 to 20 seconds in length, from 1 to 60 seconds in length, from 20 to 60 seconds in length, from 1 to 2 minutes in length, from 1 to 5 minutes in length, from 1 to 10 minutes in length, from 5 to 10 minutes in length, and from 5 to 30 minutes in length, including all time lengths in each range. In some embodiments of methods and systems of the invention, a preselected time interval is at least 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100, 110, 120, 130, 140, 150, 160, 170, 180, 190, 200, 300, 400, 500, 600, 700, 800, 900, or 1000 seconds in length, including each time length between each recited integer.

In some embodiments of methods and systems of the invention, the timing and interval length of detecting movement activity is based, at least in part, on length of an estrous cycle in the subject. For example, though not intended to be limiting, for a Peromyscus subject, which has an expected estrous cycle of 4 days, movement activity of the subject is detected continuously over a four-day period. Detected movement activities during the four-day period are measured and movement activities measured during different time periods within the four-day period can be compared. Levels of movement activities at different times during the four-day period are compared and results used to identify the reproductive state of the subject. In some embodiments, a first time period is a 24-hour time period during the four-day detection period and a control time level of movement activity is the subject's movement activity during a different 24 hour period of the four day detection period.

Some embodiments of methods and systems of the invention include detecting a subject's movement activity over a predetermined period of time and optionally repeating the detecting over one, two, three, four, or more additional time periods essentially equivalent in length to the predetermined time period. As a non-limiting example, for a Peromyscus subject, an embodiment of a method or system of the invention may include measuring movement activity of the subject or a plurality of subjects during two or more different four day periods, determining the reproductive state of the subject or subjects, respectively, in each of the four-day periods and comparing the results to assess the accuracy of the reproductive state of the subject or subjects identified in the initial four-day detection time period.

It will be understood that the length of a predetermined measuring period for a subject may be based, at least in part, on an expected length of an estrous cycle of the subject's species. In a non-limiting example, if the expected length of the estrous cycle for a member of a species of mouse that is to be a test subject is four days, the predetermined measuring period for the embodiment of the method of system of the invention used to identify the reproductive state of the test subject may be four days. In another non-limiting example, if the expected length of the estrous cycle for a member of a species of rat that is to be a test subject is four days, the predetermined measuring period for the embodiment of the method of system of the invention used to identify the reproductive state of the test subject may be four days. In another non-limiting example, if the expected length of the estrous cycle for a marmoset that is to be a test subject is 28 days, the predetermined measuring period for the embodiment of the method of system of the invention used to identify the reproductive state of the test subject may be 28 days. In another non-limiting example, if the expected length of the estrous cycle for a tree shrew that is to be a test subject is 9-12 days, the predetermined measuring period for the embodiment of the method of system of the invention used to identify the reproductive state of the test subject may be between 9 and 12 days. One skilled in the art will understand how to identify and set a predetermined measuring period based on the time length of estrus of a member of the species of test subject.

Methods and systems of the invention may include analysis of obtained movement activity measures. In some embodiments of the invention, the analysis includes performing pixel-level analysis of two or more obtained images to detect the presence or absence of differences between the two or more images. Detected differences identify movement activity by the test subject. In some instances, a level of movement activity in a test subject or plurality of test subjects is identified. As used herein in reference to movement activity, the term “level” means amount of movement activity. In some embodiments of the invention, a level of movement activity for a test subject or plurality of test subjects is identified in a first time period and in a second time period, and in some embodiments, the first and second levels are compared to identify a change in movement activity between the two time periods. A change may be an increase or a decrease in a level of movement activity. In some embodiments, a comparison of a first and second level indicates no change. A non-limiting example of identifying a level of movement activity of a test subject or plurality of subjects in a first time period includes comparing the pixel-level analysis of two or more of the images of the test subject or plurality of test subjects, respectively, obtained in the first time period.

In some embodiments of methods and systems of the invention, a level of movement activity for a subject or plurality of subjects is identified in a first time period and compared to a level of movement activity of a subject or plurality of subjects identified in a second time period. Such comparisons permit identification of a change in movement activity between the two time periods or no change in movement activity between the two time periods. If a change is identified, embodiments of methods and systems of the invention can be used to identify a level of the change in movement activity between the two time periods. In some embodiments, a first time period and a second time period may be sequential, for example in some embodiments, the second time period may occur at a time after the first time period. In some embodiments, a second time period either precedes or is subsequent to a first time period. In some embodiments of methods and systems of the invention, the length of the first time period is up to and including 24-hours. In certain embodiments of methods and systems of the invention, the length of the second time period is up to and including 24-hours. In some embodiments of methods and systems of the invention, the length of the first time period is more than 24-hours. In certain embodiments of methods and systems of the invention, the length of the second time period is more than 24-hours. It will be understood that the length of time of the first and second time periods are independently selected, meaning the length of each is selected independent of the other meaning the length of a first and a second time period may selected to be the same or the length of a first and a second time period may be selected to be different in length.

Some embodiments of methods and systems of the invention includes assessing pixels and/or machine learning (ML) methods. In some embodiments, determining a difference in a measured level of movement activity of a test subject or plurality of test subjects in a first time period compared to a control level of movement activity may include one or more of subtracting pixels and training a machine learning (ML) model. In some embodiments, the training of the ML model includes training the ML model to predict the level of movement activity of the one or plurality of subjects. In certain embodiments, training of an ML model includes training the ML model with one or more of a plurality of individual frames as inputs. In some embodiments, training of the ML model includes training the ML model with one or a plurality of a portion of a frame as an input.

### Reproductive States and Estrus-cycle Linked Activity

Methods and systems of the invention can be used to identify a reproductive state in a subject or in a plurality of subjects. It has now been determined that detected activity of a test subject or a plurality of test subjects corresponds to the reproductive state of the subject or subjects. As used herein the term “reproductive state” means a stage of the estrous cycle of a subject. The terms “estrus,” and “estrous cycle,” as used herein refer to the reproductive cycle of female subjects, which is generally defined in the art as the time from one estrus to the next. An estrous cycle includes two phases, the follicular phase (proestrus and estrus) and the luteal phase (metaestrus and diestrus). The term estrus is also referred to as a period of sexual receptivity, which may be referred to in the art as “heat.” The term anestrous means a period when a female subject does not experience cycles, non-limiting examples of situations resulting in anestrous are: pregnancy, aging, stress, when offspring are present, impaired nutrition, and pathology.

Methods and systems of the invention can be used to detect activity levels of subjects and to interpret the detected activity levels to identify a stage of the estrous cycle the subject was in at the time of the activity detection. Methods of the invention permit extrapolation of the activity detection data to identify subsequent estrus stages of a subject. As a non-limiting example, a method of the invention detects activity of a subject that indicates the subject is at a proestrus stage, and this determination can be extrapolated to identify the subject as subsequently entering estrus and ovulation stage, which follow the proestrus stage. Methods and systems of the invention includes use of cameras that detect activity changes that correlate with ovulation events, which occur during estrus. It will be understood that methods of the invention comprise detecting activity in a subject, wherein the activity (presence/absence/level) is linked or associated with the stage of the estrous cycle of the subject. Thus, activity of a test subject can be determined and identified as corresponding with a stage of the test subject's estrous cycle at the time of detection. Activity of a subject detected using a method of the invention may be referred to herein as “estrus-cycle-linked activity,” which using methods of the invention can be used to determine the reproductive state of the subject. The term estrus-state movement activities may be used interchangeable herein with the term estrus-cycle-linked activity.

In some embodiments, an identified reproductive state includes at least one of proestrus, estrus, metestrus, diestrus, anestrus, ovulation, pseudopregnancy, and pregnancy. In some embodiments of methods and systems of the invention, determining an increase in a test subject's measured movement activity in a 24-hour period compared to an adjacent 24-hour period identifies the increased activity an estrus-cycle-linked activity of the test subject, and is used to identify the reproductive state of the subject. In a non-limiting example, a method of the invention detects an increase in a plurality of test subjects' measured movement activity in a 24-hour period compared to an adjacent 24-hour period and identifies the estrous cycle stage of the plurality of subjects as ovulation.

Identification of a reproductive state of a subject or plurality of subjects may, in some embodiments of methods and systems of the invention be followed by an action that is determined based on the identified reproductive state. For example, though not intended to be limiting, identifying a test subject or plurality of test subjects with a reproductive state of estrus that is identified as ovulation, may determine an action of initiating a pregnancy in the test subject or plurality of test subjects, respectively. In another non-limiting example, identifying a test subject or plurality of test subjects with a reproductive state of estrus that is proestrus, may determine an action of initiating a pseudopregnancy in the test subject or plurality of test subjects, respectively. In another non-limiting example, identifying a test subject or plurality of test subjects with a reproductive state of estrus that is not ovulation, may determine an action of not initiating a pregnancy and/or not initiating a pseudopregnancy in the test subject or plurality of test subjects, respectively.

In some embodiments of methods and systems of the invention, identifying a reproductive state of a test subject or plurality of test subjects may determine an action comprising initiating an environmental or physical change for the test subject or plurality of test subjects, respectively. As a non-limiting example, identifying a test subject or plurality of test subjects with a reproductive state of estrus—for example identifying the test subject or plurality of test subjects as in the stage of ovulation—may determine an action of a physical change for the test subject or plurality of test subjects such as surgery and/or artificial insemination of the subject or plurality of subjects, respectively.

### Identifying Reproductive States

Differences in movement activity detected using embodiments of methods and systems of the invention can be used to identify a reproductive state of a test subject or a plurality of test subjects. In some embodiments, the absence of an increase in the measured level of movement activities of the test subject or plurality of test subjects compared to a control level of movement activities identifies the reproductive state of the test subject or plurality of test subjects as including one of: proestrus, estrus, metestrus, diestrus, anestrus, ovulation, pseudopregnancy, and pregnancy. In some embodiments, the presence of an increase in the measured level of movement activities of the test subject compared to the control level of movement activities identifies the reproductive state of the test subject as in an estrus stage that is ovulation. In some embodiments of methods and systems of the invention, the control movement activities are not estrus-state movement activities (thus are not “estrus-cycle-linked activities) and the presence of an increase in the level of the measured movement activities of the test subject or plurality of test subjects compared to the control level of movement activities identifies the reproductive state of the test subject or plurality of test subjects, respectively, as an estrus stage that is ovulation. In certain embodiments, the control movement activities are estrus state movement activities and the presence of a decrease in the level measured movement activities of the test subject or plurality of test subjects compared to the control level of movement activities identifies the reproductive state of the test subject plurality of test subjects, respectively, as not an estrus state. In some embodiments, a control movement activity includes movement activity of the test subject or plurality of test subjects that occurs in a second time period.

In certain embodiments, a control movement activity includes movement activity of the test subject or the plurality of test subjects in a time period when the test subject or plurality of test subjects, respectively, is not in estrus. In some embodiments of methods and systems of the invention, a control movement activity includes movement activity of the test subject or plurality of test subjects in a time period when the reproductive state of the test subject or plurality of test subjects, respectively, includes at least one of proestrus, metestrus, diestrus, anestrus, ovulation, pseudopregnancy, and pregnancy. In certain embodiments, a control movement activity includes movement activity measured in one or a plurality of different subjects of the same species as the test subject. In some embodiments, the one or plurality of the different subjects are not in estrus and in certain embodiments, the reproductive state of the one or plurality of the different subjects is proestrus, metestrus, diestrus, pseudopregnancy, or pregnancy.

### Testing

Methods and systems of the invention can be used to identify a reproductive state of a subject or a plurality of subjects and in some embodiments, the identification is useful to assess the presence or absence of a disease or condition, or to assess efficacy of a candidate therapeutic agent. In some embodiments, a method or system of the invention includes administering a candidate therapeutic agent to a test subject or plurality of test subjects, at least one or more of: before, during, and after the measuring of movement activity in the test subject or plurality of test subjects, respectively. In some embodiments, the method or system also includes monitoring the subject or plurality of subjects and identifying one or more of a presence, an absence, and a level of an effect of the administered candidate therapeutic agent on the test subject or plurality of test subjects.

### Subjects

Some aspects of the invention include methods to identify a reproductive state of a subject. As used herein, the term “subject” may refer to a human, non-human primate, cow, horse, pig, sheep, goat, dog, cat, pig, bird, rodent, or other suitable mammalian organism. In certain embodiments of the invention, a subject is a mammal and in certain embodiments of the invention, a subject is a rodent, including but not limited to a: mouse, rat, gerbil, hamster, etc. In certain embodiments, methods of the invention are used with a captive subject, non-limiting examples of which are caged research animals, zoo animals, farm animals, domesticated animals, etc. In some embodiments of methods and systems of the invention, a subject is a rat that is a member of a refractory species of rat, a prairie vole, a spiny mouse, a tree shrew, a marmoset, or a naked mole rat. In some embodiments, methods of the invention are used in wild species of subjects, non-limiting examples of which are subjects in the wild or subjects obtained from the wild, either directly or as a descendent of a captive wild subject. In some embodiments, a subject is a household pet, a service animal, a working animal, a farm animal, a research animal, a feral animal, an animal naturally in the wild, an animal that has been released into the wild, an animal in a care environment, a non-limiting example of which is a zoo animal, an animal in a rehabilitation environment, etc. In some embodiments of the invention, a subject is a normal, healthy subject and in some embodiments, a subject is known to have, to be at risk of having, or to be suspected of having a disease or condition. The terms “subject” and “test subject” may be used interchangeably herein.

A subject for which a reproductive state may be identified using embodiments of methods or systems of the invention is a female subject that is of reproductive age. The term “reproductive age” means the subject is mature to the degree at which members of the subject's species are considered sexually mature. It will be understood that a sexually mature subject may or may not be capable of reproducing depending on factors such as, but not limited to a physical condition of the subject, the health of the subject, an intervention with the subject's ability to reproduce, etc. Non-limiting examples of a subject not considered to be of reproductive age is an young animal not yet sexually mature, an animal beyond its reproductive age, or an animal that lacks reproductive capacity, for non-limiting reasons such as: a medical event, an environmental event, a surgical event, a disease, an inherited characteristic, an accident, etc., that eliminated the subject's reproductive capacity.

As a non-limiting example, a subject assessed with an embodiment of a method of system of the invention is a subject that is an animal model for a disease or condition. Non-limiting examples of diseases and conditions are reproductive diseases or conditions, neuromuscular diseases or conditions, a disease or condition associated with inflammation, a post-operative condition, a cancer condition, a sensory condition, a genetic disease, or condition. Additional animal models of diseases or conditions suitable for use in methods and systems of the invention are known in the art.

In some embodiments of methods and systems of the invention, a subject is a wild-type subject. As used herein the term “wild type” means to the phenotype and/or genotype of the typical form of a species as it occurs in nature. In some embodiments, a wild-type subject is in or is obtained from a wild environment. As used herein the term “wild environment” means the environment is not a captive environment. In certain embodiments of the invention a subject is a non-wild-type subject, for example, a subject with one or more genetic modifications compared to the wild-type genotype and/or phenotype of the subject's species. In some instances, a genotypic/phenotypic difference of a subject compared to wild-type results from a hereditary (germline) mutation or an acquired (somatic) mutation. Factors that may result in a subject exhibiting one or more somatic mutations include but are not limited to environmental factors, toxins, ultraviolet radiation, a spontaneous error arising in cell division, a teratogenic event such as but not limited to radiation, maternal infection, chemicals, etc.

In certain embodiments of methods of the invention, a subject is a genetically modified organism, also referred to as an engineered subject. An engineered subject may include a pre-selected and/or intentional genetic modification and as such exhibits one or more genotypic and/or phenotypic traits that differ from the traits in a non-engineered subject. In some embodiments of the invention, routine genetic engineering techniques can be used to produce an engineered subject that exhibits genotypic and/or phenotypic differences compared to a non-engineered subject of the species. As a non-limiting example, a genetically engineered mouse in which a functional gene product is missing or is present in the mouse at a reduced level and a method or system of the invention can be used to assess the genetically engineered mouse phenotype, and the results may be compared to results obtained from a control (control results).

### Controls and Candidate Compound Testing and Screening

Results obtained for a subject using an embodiment of a system or method of the invention can be compared to control results. Methods of the invention can also be used to assess a difference in a reproductive state in a subject or subjects versus a control. Thus, some aspects of the invention provide methods of determining the presence or absence of a change in an activity in a subject compared to a control. Some embodiments of the invention include using an embodiment of a system or method of the invention to identify a reproductive state associated with a disease or condition and in certain embodiments of the invention can be used to assess an effect of a candidate therapeutic compound on a subject or subjects.

Results obtained using a method or system of the invention can be advantageously compared to a control. In some embodiments of the invention, one or more subjects can be assessed using a method or system of the invention followed by assessing the subject or subjects following administration of a candidate therapeutic compound to the subject(s). The terms “subject” and “test subject” may be used herein in relation to a subject that is assessed using a method or system of the invention, and the terms “subject” and “test subject” are used interchangeably herein. In certain embodiments of the invention, a result obtained using a method or system to assess a test subject is compared to results obtained from the methods performed on other test subjects. In some embodiments of the invention, a test subject's results are compared to results of the methods of the invention performed on the test subject at a different time. In some embodiments of the invention, a result obtained using a method or system of the invention to assess a test subject or subjects is compared to a control result.

As used herein a control result may be a predetermined value, which can take a variety of forms. It can be a single cut-off value, such as a median or mean. It can be established based upon comparative groups, such as subjects that have been assessed using a method or system of the invention under similar conditions as the test subject, wherein the test subject is administered a candidate therapeutic agent and the comparative group has not been contacted with the candidate therapeutic agent. Another example of comparative groups may include subjects known to have a disease or condition and groups without the disease or condition. Another comparative group may be subjects with a family history of a disease or condition and subjects from a group without such a family history. A predetermined value can be arranged, for example, where a tested population is divided equally (or unequally) into groups based on results of testing. Those skilled in the art are able to select appropriate control groups and values for use in comparative methods of the invention.

A subject assessed using a method or system of the invention may be monitored for the presence or absence of a change that occurs in a test condition versus a control condition. As non-limiting examples, in a subject, a change that occurs may include, but is not limited to one of more of: a level of movement activity, etc. Methods and systems of the invention can be used with test subjects to assess the effects of a disease or condition of the test subject and can also be used to assess efficacy of candidate therapeutic agents. As a non-limiting example of use of method of the invention to assess the presence or absence of a change in a test subject as a means to identify efficacy of a candidate therapeutic agent, a test subject known to have a reproductive state-associated disease or condition is assessed using a method of the invention. The test subject is then administered a candidate therapeutic agent and assessed again using the method of the invention. The presence or absence of a change in the test subject's results indicates a presence or absence, respectively, of an effect of the candidate therapeutic agent on the disease or condition.

It will be understood that in some embodiments of the invention, a test subject may serve as its own control, for example by being assessed two or more times using a method or system of the invention and comparing the results obtained at two or more of the different assessments. Methods and systems of the invention can be used to assess progression or regression of a disease or condition in a subject, by identifying and comparing changes in a reproductive state in a subject over time using two or more assessments of the subject using an embodiment of a method or system of the invention.

### EXAMPLES

### Example 1

### Materials and Methods

### Superovulation of Peromyscus.

All mouse experiments were performed according to NIB and DOD guidelines and approved by the MIT Committee on Animal Care. Briefly, 4 to 6-week-old female P. leucopus were injected intraperitoneally with 20 IU of PMSG 1 hour prior to lights out followed by 20 IU of hCG 72 or 48 hours later. Mice were mated overnight and 36 hours after timed matings were set up, embryos were collected from donor females after CO₂ euthanasia.

### De Novo Sequencing of Peromyscus Genome.

Blood samples were collected in a collection tube coated with an anticoagulant (EDTA) and inverted 8-10 times immediately following collection to ensure proper mixing of the anticoagulant. Blood was shipped on dry ice immediately following extraction to Dovetail Genomics (Santa Cruz) for de novo genome assembly.

### Guide Targeting Construct Design & Guide Design.

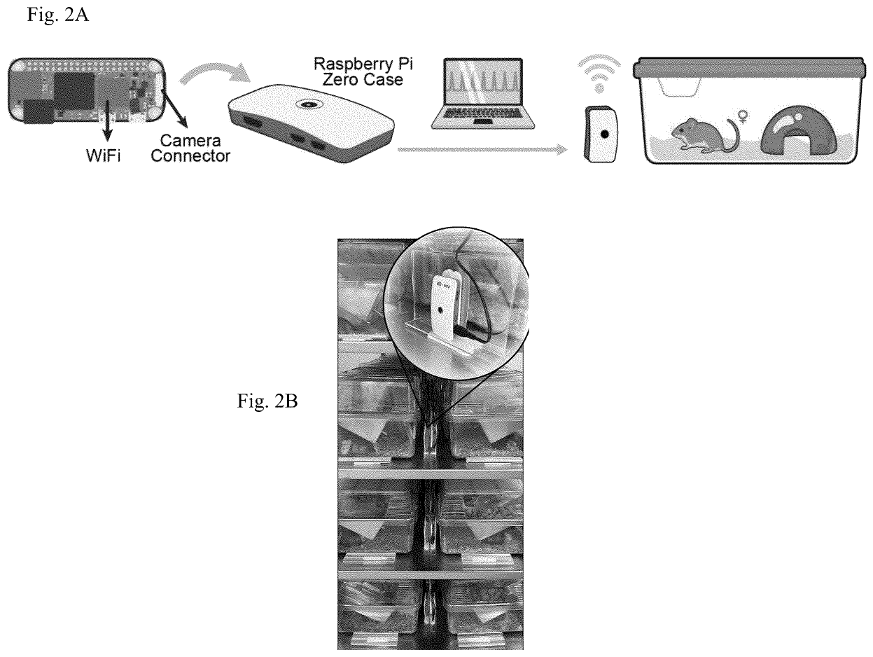

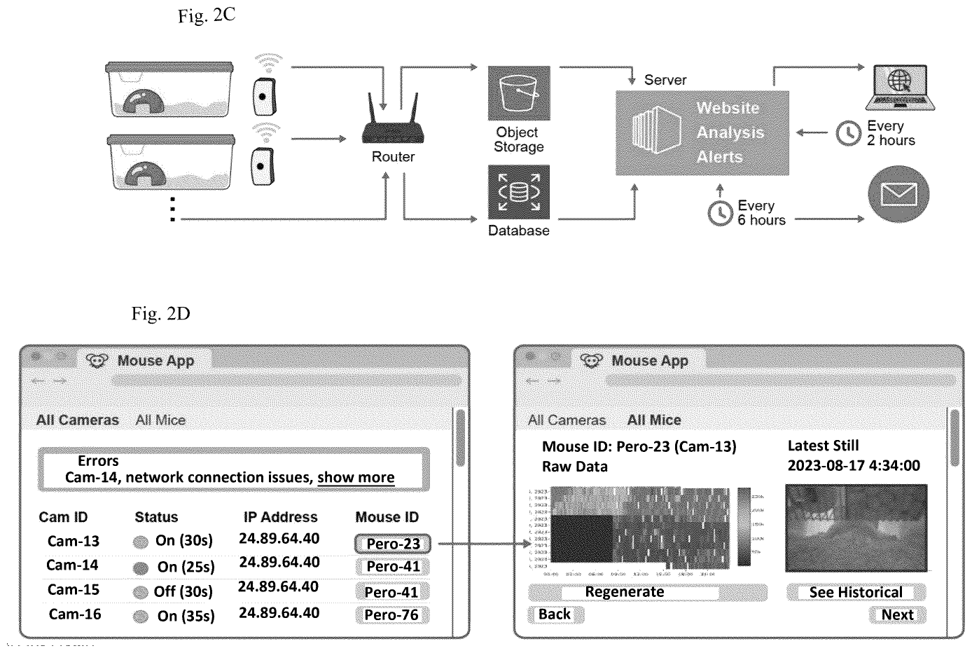

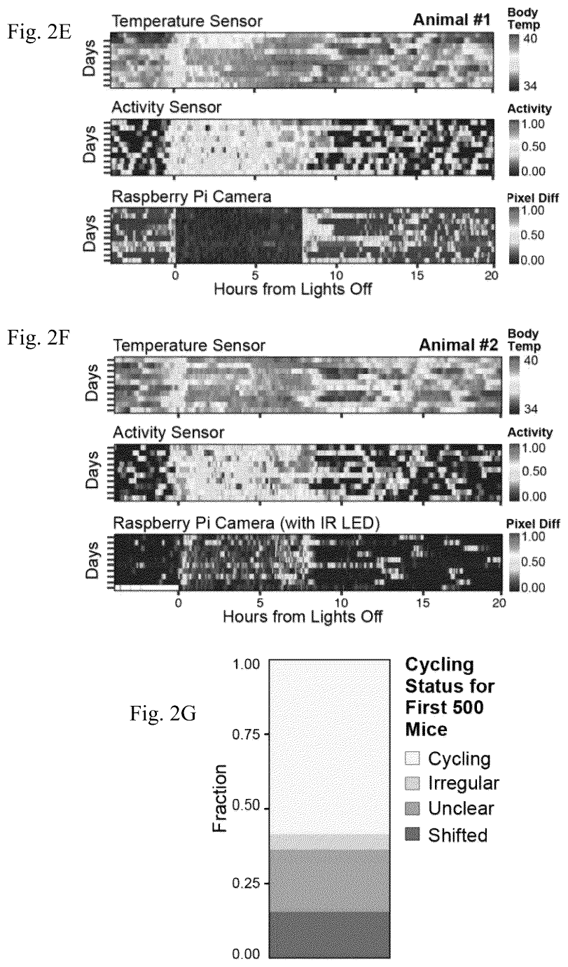

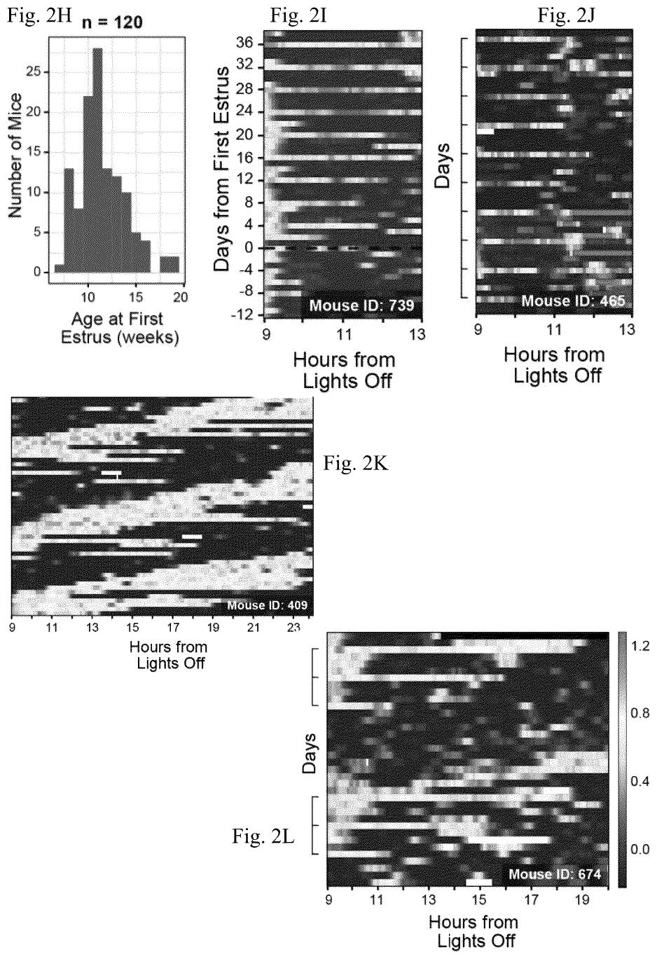

Rosa26 sgRNA for iGONAD and pronuclear injection was purchased from Integrated DNA Technologies (FIG. 2A). Rosa26 homology arms for ssDNA (for iGONAD) and targeting constructs (for pronuclear injection) were identified by aligning the Mus Rosa26 locus to the Peromyscus genome assembled via Dovetail Genomics. Homology arms were synthesized as gBlocks via IDT and either cloned into a standard high-copy number plasmid backbone, or used to make ssDNA, which was produced using the Guide-it Long ssDNA Production System v2 from Takara.

Electroporation of pESCs.

1×10⁷ cells were electroporated with 20 mg of DNA using a Lonza nucleofector and then seeded on MEFs.

### Karyotyping.

Live cells at passage 15 were sent to Cell Line Genetics for cytogenetic analysis. Analysis was performed on twenty G-banded metaphase male pESCs. Two abnormal male clones were analyzed. Clone 1 contained eighteen cells with trisomy 16 and trisomy 17. Clone 2 contains 2 cells which demonstrated trisomy 16.

i-GONAD.

Mouse oviduct electroporation was performed as previously reported [Ohtsuka et al., Genome Biology, 19(1), 25 (2018)]. Briefly, 8-16 week old Peromyscus were mated with stud males and checked daily for sperm using vaginal lavage. If sperm was found, the female was subjected to iGONAD the same day. CRISPR gene editing cocktails were freshly assembled and contained 6 μM Cas9 protein (IDT 1081058, Lot #0000405530), 30 μM gRNA, and 18 μM ssODNA (produced in-house). This cocktail was delivered into the oviduct of female mice through microcapillary injection. Oviduct electroporation was performed using a NEPA 21 Electro-Kinetic Transfection System with a range of conditions including: Pd A: between 100-135 mA, Pd on: 5 msec, Pd off: 50 msec, three cycles, decay 10%.

### Genotyping.

The screening of founder pups was performed by PCR and direct sequencing using DNA obtained from ear slices. Genomic DNA was extracted from ear slices with the GenElute Mammalian Genomic DNA Mini-prep Kit (Sigma-Aldrich). PCR was performed with PrimeSTAR® DNA Polymerase (Takara Bio) and the following primers Rosa26-F (5′-gggagttctctgctgcct-3′ SEQ ID NO: 1) and Rosa26-R (5′-agttttgctgcataaaacccc-3′ SEQ ID NO: 2).

### Temperature & Activity Data Monitoring.

Data were gathered with G2 E-Mitter implants for core body temperature and locomotor activity (Starr Life Sciences Corp., Oakmont, PA). G2 E-Mitters were implanted in the abdominal cavity under anesthesia using a cocktail containing ketamine (150 mg/kg), xylazine (5 mg/kg) and acepromazine (2 mg/kg). E-Mitters were sutured to the ventral muscle wall to maintain consistent core temperature measurements. Recordings began immediately, but mice were not mated until at least 3 cycles of regular estrus were observed. Recordings were collected continuously at 1-min resolution for core body temperature and activity. All mice were between 10 and 16 wk of age at the time of implant surgery and were handled once/wk across recordings at the time of cage changes, but otherwise were left undisturbed in single housing.

### P. Leucopus Mating Set-up in Estrous Tracking Experiments.

Animals were maintained under 16:8 LD cycle of ~400 lux (light) to <1 lux red light (darkness), with lights on from 4 am to 8 pm. Food and water were available ad libitum. Mouse temperature and activity profiles were utilized to monitor estrous cycles remotely and mice were paired on the day of apparent estrus from 1 hr before lights-off (7 pm) to 5 h after lights-on the following day (9 am). Vaginal plugs were not monitored. Removal of the male was the only disturbance to the females.

Mus musculus Estrous Tracking Experiments.

8 week old Mus musculus (C57BL/6) were maintained under 15:9 LD cycle of ~400 lux (light) to <1 lux red light (darkness), with lights on from 6 am to 9 pm. Food and water were available ad libitum. Mus activity profiles were used to monitor estrous cycles remotely. Cage changes were the only disturbance to the animals.

### Hamster Estrous Tracking Experiments.

14 week old Syrian hamsters Mesocricetus auratus were maintained under 12:12 LD cycle of ~400 lux (light) to <1 lux red light (darkness), with lights on from 7 am to 7 pm. Food and water were available ad libitum. Hamster activity profiles were used to monitor estrous cycles remotely. Cage changes were the only disturbance to the animals.

### Camera Setup, Data Export & Analysis.

Each Raspberry Pi Zero 2 W was assigned a unique ID used to organize the data it processed. Each drive had a capacity of 32 GB and was flashed with the Raspbian OS image and a script for startup configuration. The Raspberry Pi NoIR Camera V2 modules were connected to the Raspberry Pi Zero 2 W using a ribbon cable, and both were assembled into the official Raspberry Pi Zero case. System monitoring and alerting was done using DataDog and PagerDuty, respectively.

The Raspberry Pi systems were attached to acrylic stands using Command strips for ease of repositioning. The stands were placed next to each cage. For one experiment, near-IR (940 nm), 100 mA LEDs were attached to the stands and powered by the Raspberry Pi USB port to illuminate the mice at night.

Linux' systemd utility was used to manage the image capture, processing and upload services. The image capture service captures 640×480 RGB images every 4 seconds using raspistill and saves them locally as jpegs. Every 10 minutes, the image processing service runs a Python program that uses OpenCV to compare each 2-tuple of consecutive images using the imagediff algorithm. Next, this service attempts to upload this data to a Postgres database in AWS RDS, then deletes the processed images to save storage. To mitigate network reliability issues, a copy of the data is written to a local file, to be recovered when the connection is restored. If enabled, the image upload service uploads stills to AWS S3, and inserts a corresponding row referencing the S3 URI into the database. Stills are stored until a successful upload, or until the disk reaches capacity.

All Raspberry Pis were connected to a VPN running on an AWS EC2 instance for remote debugging and to receive over-the-air software updates; other data was not sent over the VPN. Pis uploaded their IP address to S3 every 2 minutes, in part serving as a health check.

An AWS EC2 instance ran an alerting system as well as a web server for monitoring and data visualization. Every 6 hours, a Python script checked that each Pi has uploaded its IP and data recently, and sent an email listing the Pis that have not. Every hour, the Raspberry Pi ID to mouse ID mapping was read from a Google Sheet and inserted into the database. Data was then downloaded from the database per mouse, grouped into 5-minute windows, and stored in a day by window format. Further data processing in the form of outlier removal was available interactively on the web interface.

### Vasectomies.

Briefly, 8-12 week old male Peromyscus were anesthetized with a cocktail containing ketamine (150 mg/kg), xylazine (5 mg/kg) and acepromazine (2 mg/kg) in preparation for surgery. Once anesthetized, the eyes were covered with ointment to prevent dryness and the abdomen was shaved with small clippers and cleaned with betadine followed by 70% ethanol, 3 times. Following aseptic technique, a small incision of about 1.5 cm was made at a point level with the top of the legs. Another small incision was made in the body wall along the linea alba. With blunt forceps, the fat pad was pulled out, exposing the vas deferens. The vas deferens was cauterized with a high temperature cautery power handle. The fat pad was then reinserted inside the body wall, and repeated on the other side. The body wall was sutured with 1-2 stitches of coated VICRYL sutures and the skin was closed with wound clips which were removed 7-14 days later.

### Embryo Transfers.

Briefly, 8-16 week old female Peromyscus were mated with vasectomized males on the night of apparent estrus to generate pseudopregnant recipients. The following day, females were anesthetized with a cocktail containing ketamine (150 mg/kg), xylazine (5 mg/kg) and acepromazine (2 mg/kg) in preparation for surgery. Once anesthetized, the eyes were covered with eye ointment to prevent dryness and the lower back of the recipient mouse was shaved with small clippers. Using cotton tip applicators, the surgical area was cleaned with betadine followed by 70% ethanol, repeated 3 times. Following aseptic technique, a small transverse incision (less than 1 cm) was made with fine dissecting scissors about 1 cm to the right of the spinal cord at the level of the last rib. Once the ovary or fat pad could be visualized through the body wall, a small incision was made just over the ovary. With blunt fine forceps, the fat pad was pulled through the incision and the ovary, oviduct and uterine horn was pulled out and laid down over the middle of the back, so that the oviduct was exposed outside the body wall. Manipulated 2-cell embryos were transferred into the oviduct by puncturing the oviduct wall, in between the infundibulum and ampulla with a sterile 30 g needle and transferring the embryos through the small opening. Once the embryos have been transferred, the ovary, oviduct and uterus were placed inside the body wall. The body wall was sutured with 1 or 2 stitches of coated VICRYL sutures and the skin was closed with wound clips, which were removed 7-14 days after embryo transfer.

### Pronuclear Injection.

Briefly, Peromyscus females were used as embryo donors and recipients. Camera-tracked donors were mated with proven studs. Two days later, 2-cell embryos were flushed from the oviducts using prewarmed M2 medium. After embryo collection, 2-cell embryos were transferred to pre-equilibrated KSOM (CytoSpring; K0102) overlaid with mineral oil and incubated at 37° C. (5% CO₂ and 5% O₂). SpCas9 protein (final concentration 50 ng/μl) and sgRNA (final concentration 50 ng/μl) were mixed and incubated at room temperature for 15-20 minutes. DNA template was added to the RNP solution at a final concentration of 10 ng/μl. The pronuclei of each blastomere was injected for each 2-cell before incubating the embryos in KSOM. Pressure for microinjection was provided by Eppendorf FemtoJet 4i. Peromyscus females were mated to vasectomized Peromyscus males one day prior to 2-cell microinjection. The same day as microinjection, manipulated 2-cell embryos were transferred to pseudopregnant females' oviducts.

### Image Processing for Mus and Hamsters.

Still images captured every 4 seconds with a resolution of 640×480 were cropped to retain only the quadrant where food intake activities were observed. For Mus, only images from 0600-0900 UTC each day were used for downstream analysis. For hamsters, only images from 0000-0820 UTC were used. Each frame was then normalized to have a minimum of 0 and maximum of 255 in each of the 3 channels. “TwoPoint” background subtraction algorithm from Python package bgslibrary was then applied iteratively to each frame and a background learned and subtracted from each frame. These frames were then normalized to a range of 0 to 1, and L2 norms computed for each frame and stored in a separate array for each day. To reduce the impact of spurious activities, adjacent elements in this array above a threshold of T but forming blocks within the index range of (p,q) of less than N or more than N′ neighbors were replaced with the mean of elements with indices p-1 and q+1. For Mus, T=25, N=20, N′=200. For hamsters, T=15, N=3, N′=100. The differences between adjacent elements of this resulting array was then computed for each day and plotted as a heatmap. For heatmap visualization, a max and min normalization value was set using 30 break histogram binning. More specifically, a 30 bin histogram of the log 10 difference was generated with 0 and 1 set just outside the tails. For the cumulative activity plots, activity levels for each time point were convolved with a kernel of one hundred ones, max pooled with a block size of 100 and the daily sum was plotted as time series bar plots. Autocorrelation of daily activity levels was then computed to identify periodicity with a 95% confidence interval.

### Thermal Activity Tracking.

Still images were captured using the FLIR Lepton 3.0 (Teledyne FLIR, Wilsonville, OR) attached directly to a Raspberry Pi Zero 2 W computer or to a PC via a PureThermal 3 adapter (GroupGets, Reno, NV). An IR-transparent zinc selenide disk was used to construct an imaging window for the thermal camera. Images were sectioned according to the Image Processing For Mus and Hamsters section above. Cropped images were then binarized and the difference between the L2 norms of two adjacent frames was used as a proxy of activity for downstream analysis. Again for heatmap visualization, a max and min normalization value was set using 30 break histogram binning. For the cumulative activity plots, activity levels for each time point were convolved with a kernel of one hundred ones, max pooled with a block size of 100 and the daily sum was plotted as time series bar plots. Autocorrelation of daily activity levels was then computed to identify periodicity with a 95% confidence interval.

### Vaginal Swabbing, Cytology & Analysis.

A cohort of 96 Peromyscus was monitored via camera to determine their ovulation timing. Every 6 hours starting from midnight on the night of ovulation, vaginal samples were collected from groups of 4 Peromyscus using sterile nasopharyngeal nylon flocked swabs moistened with 1× sterile PBS. The swabs were inserted roughly 2 mm into the vagina, rotated times, then the sample was transferred onto individual slides (one per mouse) and left to dry. Slides were fixed by submerging in methanol for 45 seconds and air-dried. Following fixation, slides were stained with the Epredia Three-Step Stain Kit (Epredia, Kalamazoo, MI). TissueFAXS Plus slide scanner at 10× magnification under brightfield was used to image entire slides, generating hundreds of images per slide/mouse. For analysis, 3 highly confluent images from each slide/mouse were selected; cells within these images were counted using the Cell Counter plugin in ImageJ, and classified as cornified, leukocyte, or nucleated. The average count of each cell type across all 3 images was calculated and graphically represented against the sample collection time.

### Machine Learning Using Vaginal Cytology.

120 Peromyscus mice were monitored via camera tracking to pinpoint their ovulation timings. These mice were evenly divided into four groups, with members from each group swabbed daily at 9 am until each of the 120 mice had been sampled once. Sample processing and scanning adhered to the procedure outlined in the previous Vaginal Swabbing section. Subsequently, a total of 12,000 images were split into training and validation sets at an 80% to 20% ratio. Pre-trained ResNet-50 model was fine-tuned for 20 epochs and the best model weights were saved for prediction. Twelve mice unseen by the model during training were then used as test data to evaluate model performance.

### Peromyscus Perfusion, Tissue Processing and Histology.

A cohort of old female mice (133 to 155 weeks old) were placed on camera and monitored until cycling activity could be confirmed. Three cycling and three non-cycling mice were chosen for further histological analysis. The reproductive system of mice that appeared to be cycling were collected on the day of presumed estrus.

Briefly, mice were anesthetized with isoflurane and perfused transcardially with phosphate buffered saline (PBS) as previously described [Gage, G. J., et al., J. Vis. Exp. (2012) doi:10.3791/3564]. Vagina/uterus were immersed fixed in 10% neutral buffered formalin, routinely processed, paraffin embedded, sectioned at 4 μm and stained with hematoxylin and Eosin (H&E). Slides contained cross sections of the uterine horns and a longitudinal section of the distal uterus with adjacent conjoined cervix and vagina as per Johnson, J., et al., The Laboratory Mouse: A Guide to the Location and Orientation of Tissues for Optimal Histological Evaluation (1st ed.). CRC Press. https://doi.org/10.1201/9780429057755. Ovaries were embedded in Tissue-Tek® O.C.T. Compound, frozen on dry ice, and step-sectioned through the specimen at 100 μm intervals. Each resulting 6 pm section was manually H&E stained.

All slides were digitally captured using the Aperio AT2 scanner (Leica Biosystems, Buffalo Grove, IL) at 20× magnification and images were saved in .sys format.

The Peromyscus estrous cycle staging and evaluation of the female reproductive system for evidence of reproductive aging was performed by a board-certified veterinary pathologist based on published literature for rodents [Dixon, D. et al. J. Toxicol. Pathol. 27, 1S-107S (2014); Chanut, F. J. A. & Williams, A. M., J. Toxicol. Pathol. 44, 43-50 (2016); and Westwood, F. R., J. Toxicol. Pathol. 36, 375-384 (2008)].

### Histological Analysis.

The histology of the estrous cycle in Peromyscus, as well as information on Peromyscus reproductive aging, has not been previously published. Therefore, interpretation of Peromyscus reproductive histology is primarily based on extensive literature on rats, with additional insights from fewer studies on mice and hamsters.

Estrous Cycle Evaluation for Each Mouse in this Study:

Mouse IDs: 1122, 1047 and 1118 were in estrus. Changes in the ovaries were characterized by large antral (F2) follicles, newly formed small basophilic corpora lutea (CL) and prominent eosinophilic CL with luteal cell apoptosis from previous cycles. Uterus consistently had a tall and columnar epithelium with low number of scattered epithelial apoptotic cells and luminal dilation. Within the vagina, visible shedding of the cornified layer was present along with sloughed cornified cells and debris in the lumen.

Mouse ID: 1117 had Persistent Estrus with signs of ovarian aging characterized by many atretic follicles and lack of corpora lutea. The uterus displayed early signs of endometrial hyperplasia and there was cornification of the vaginal epithelium.
Mouse ID: 1116 had Persistent Diestrus with signs of ovarian aging showing no new CL and very few CL, most of which were atretic follicles. The endometrial epithelium was low cuboidal and there were some areas of mucification of the vaginal epithelium.

Signs of ovarian aging: The ovary contained few new follicles (approaching complete follicular depletion) and all appeared atretic. There was an absence of new corpus lutea (CLs) with basophilic cytoplasm, and an overall reduced CL count [Westwood, F. R. J. Toxicol. Pathol. 36, 375-384 (2008) and Ovary-Atrophy. Nonneoplastic Lesion Atlas. ntp.niehs.nih.gov/atlas/nnl/reproductive-system-female/ovary/Atrophy (cited April 2024), for signs also seen in mice and rats].

Signs of persistent diestrus in the uterus: As with persistent diestrus in rats, the endometrial epithelium was low, cuboidal, and inactive [Westwood, F. R. J. Toxicol. Pathol. 36, 375-384 (2008)].

Signs of persistent diestrus in the vagina: The vaginal epithelium appeared low and somewhat mucified, consistent with observations of persistent diestrus in rats [Westwood, F. R. J. Toxicol. Pathol. 36, 375-384 (2008)].

Mouse ID: 1048 is described as a mouse with an Aging Pathology characterized by a small ovary nearing anestrus with very few atretic follicles and abundant pigmented stromal tissue. The uterus had evidence of endometrial cystic hyperplasia with concurrent pyometra characterized by numerous dilated glands filled with hemorrhage, proteinaceous material and numerous degenerated neutrophils and cell debris. Numerous neutrophils were also present within the endometrial stroma. The vaginal epithelium was cornified with sloughed cornified cells and degenerated neutrophils, cell debris and hemorrhage within the lumen.

Signs of ovary nearing anestrus: As observed in mice and rats, the ovary was smaller and contained few follicles, with near-complete follicular depletion. All follicles were atretic, precluding ovulation. Furthermore, an absence of corpus lutea and an increase in stromal tissue with prominent pigment in the interstitium were noted, indicative of aging in mice and rats Westwood, F. R., J. Toxicol. Pathol. 36, 375-384 (2008) and Ovary-Atrophy. Nonneoplastic Lesion Atlas. ntp.niehs.nih.gov/atlas/nnl/reproductive-system-female/ovary/Atrophy (cited April 2024), for all signs]. Notably, this mouse presented with pyometra. However, pyometra/inflammation is not believed to be the cause of ovarian atrophy. Pyometra is an inflammatory condition of the uterus that may arise when the normal flora becomes opportunistic under certain conditions. The observed ovarian atrophy likely resulted from ovarian exhaustion due to hormonal dysregulation associated with aging.

Signs of aging in the uterus: Reproductive tract pathology confirmed the mouse's non-cycling status, with histologic evidence of endometrial cystic hyperplasia and pyometra. In aging mice and rats, endometrial cystic hyperplasia disrupts the estrous cycle and can lead to a phenotype of persistent estrus through hormonal dysregulation [(Dixon, D. et al. J. Toxicol. Pathol. 27, 1S-107S (2014)].

Signs of aging in the vagina: The morphological features of the vagina provided evidence of hormonal dysregulation or persistent estrus, characterized by a visibly shedding cornified layer. Additionally, the presence of numerous degenerated neutrophils and red blood cells in the lumen indicated inflammation.

### Homology Arm Sequences for Targeting Constructs

5′ Rosa26 Homology Arm
(SEQ ID NO: 3)
ggtggtcagggccatggtccgccctgccgcaaccggaggggggggaggag
ggagcggaaagatctccaccggacgcggccatggctcccacggggggcgg
aggagcgcttccggttcccgtctcgtcgctgattggccttttctcctccc
gccgtgtgtaaaaacacaaatggcgtgttttggttggagtgaagcgcctg
tcagttaacgggtgctggagtgcgcagccgctggctgcctcgctgtgccc
actgggtggggcgggaggtaggtggggtgaggagagctggacgtgcgggc
gcggccggcccctggcgggggggggaagggaggggagggagggtcagcga
aagtggctggcgcccaggcggcctcccaccctccccttcctctgggggag
tcggtttacccgccgccggcctggcctcgtcgtctgattggctcccgggg
cccagaaaactggcccctgcaattggcccgcgttcgtgcaagttcagtcc
atacgcgggctggcgggggcggcagggaggcgctcgcaggttccggccct
ccccaaggccccgcgccgcagagtctggccccgcgcccctgcgcaacgtg
gcaggaagcgcgcgctgggggggggacgggcaggcggtctgagcggcggg
ggggggcgagcgcggttcctcctagagttgttgccgcacgaggggcgagc
tgaaccggacctgcatggcgcactcctaggagtggaggaaggagtggggg
ctcagtcgggccggtttggaggcaggaggcacttgctctccaaaattccg
tgagagctgcaatcaataagggagccgcagtggagtaggggggagaaggc
cgcacccttctcggcagggggaggggagtgccgcaatacctttctgggag
ttctctgctgcctcctggcttctaaagaccgccccgggcctggaacagtc
ccttccccctctcccctcgtgatctgcaagtcgaggctttctgga.
3′ Rosa26 Homology Arm
(SEQ ID NO: 4)
agatgggcgggagtcttctaggctggcttaagggctaacctggtgcgtgg
gcgttgtcctgcaggggaattgaacaggtttaaaattggagaaacgacac
ttcccacagattttcgtttgtgtccggagggaattgtaataggagcaaag
gagggaaatgggtgactaggcgctcacctggggttttatgcagcaaaact
acaggttattattgcttatgatctgccttgaagtatttttcatcgtgtta
gattaaagacatgctcacccaagtttatactcccacttgagatccttgtt
atatcatgaaattttagtatcgtaaattagaatatataaacagaatctta
gcagtttctgcagagcccagtgcttcactctgtccctcttgctccctctg
cagccctaccaagagatattttagcgctctctcattttagtcccgttttc
atttgttgtactggcttatccaatccctagacagagcactggcattcttc
ctctcctgatcttagaagcctgatgagtcatgaaaccagacagattagtt
acaccacaaattgaggctgtagctagggccttgccctgcagttcttttat
tcctccttagtacattttgttgactgtttgccttgatttcattttctatc
cccacccttcaggagctttactacaatagatttattgagaatctaaccta
aagttcagcttctttccactcccatgtatgcctatctcctttttcaccat
taataaactgaactgttattaatggcttccagatggatgtctcctccaat
atcacctgatgtatcttacatatcgtcaggctgattgtattaaaaggtac
atgaattgacatgaaatttactgactccttaatgctttagtggattccat
gagtacaatacagaagaccgttaatgggctactgacagctttttcatttc
attatttcctgcatacacactttgagtcacatggaattgattactgctta.

### Studies and Results

In Situ Germline Genome Engineering Via i-GONAD

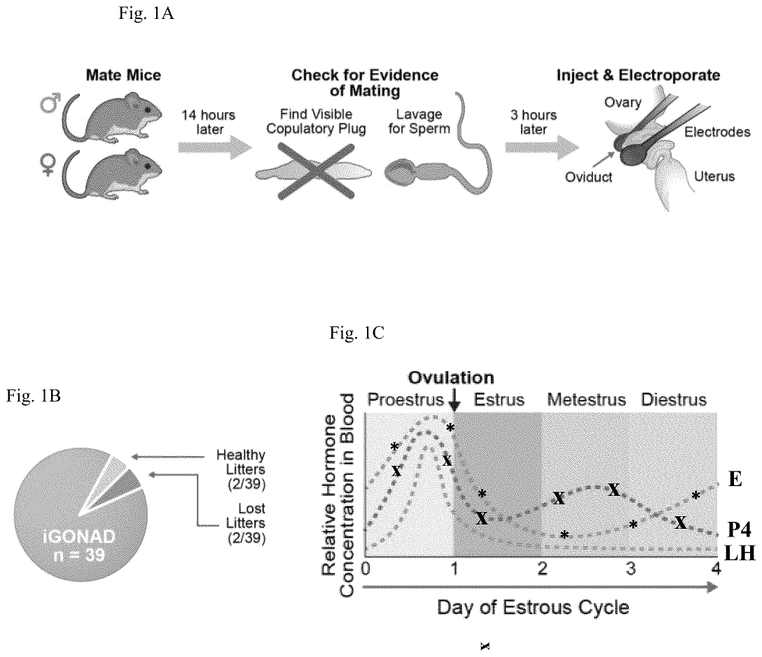

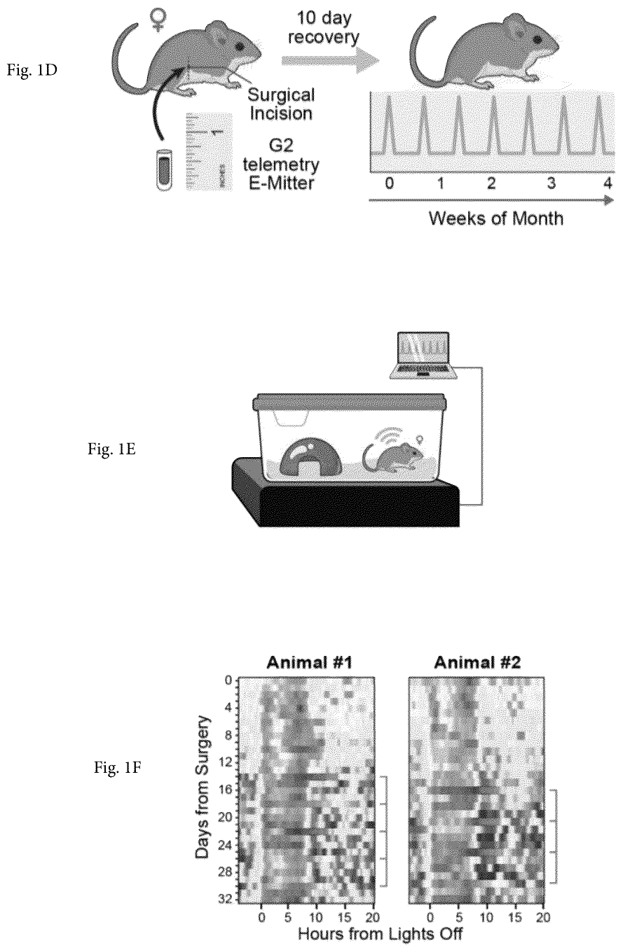

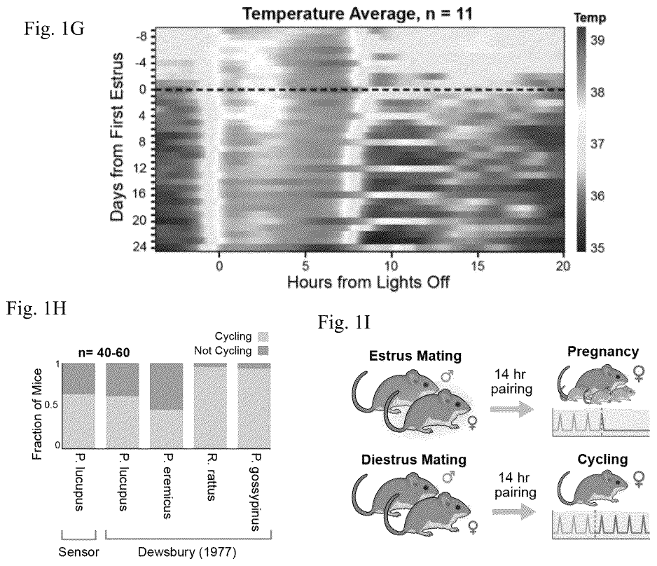

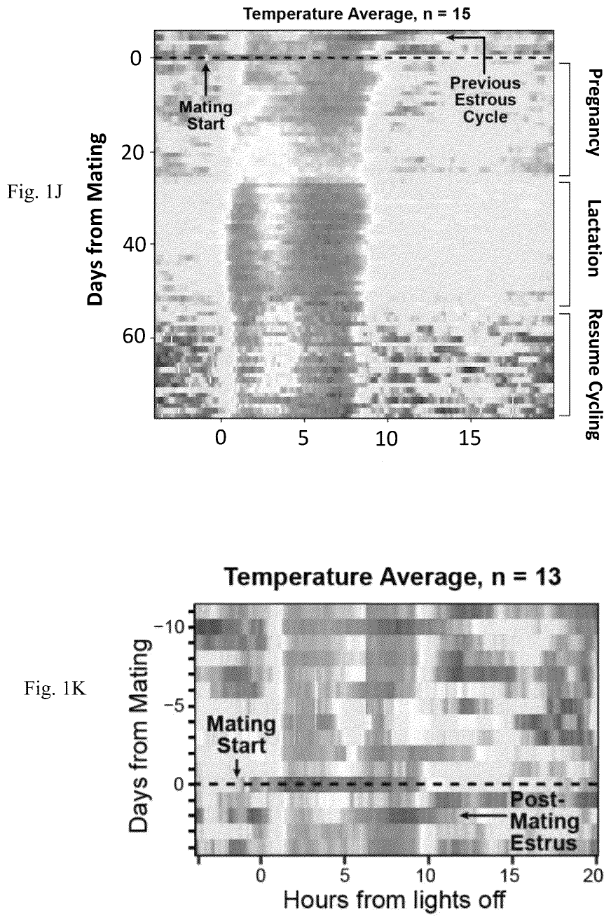

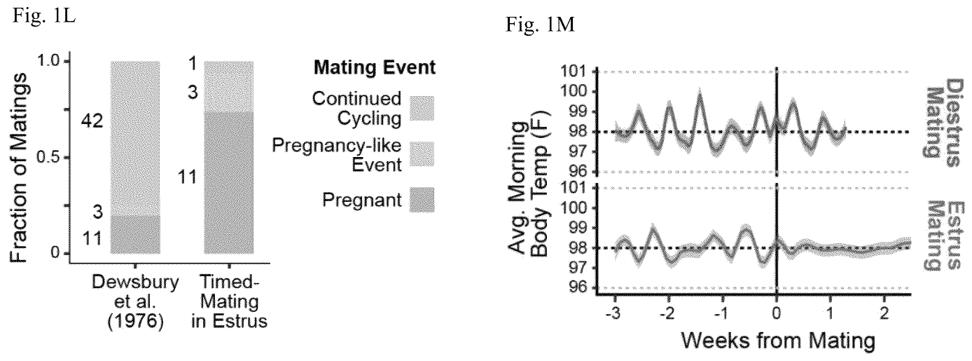

Studies were performed that included directly engineering Peromyscus embryos using i-GONAD, an electroporation method for generating germline-genome engineered animals which has been successfully implemented in multiple rodent species (FIG. 1A) [Ohtsuka et al., Genome Biology, 19(1), 25 (2018)]. First, the intraoviductal injection and electroporation technique was validated by delivering EGFP mRNA into preimplantation embryos via in situ electroporation. Fluorescence was observed in recovered embryos, confirming successful mRNA delivery via i-GONAD. Studies were performed to engineer Peromyscus embryos with i-GONAD by delivering CRISPR ribonucleoproteins, including previously validated Peromyscus Rosa26 CRISPR guide, into the oviducts of female Peromyscus via in situ electroporation. 39 i-GONAD procedures were performed on previously mated female Peromyscus, which gave rise to 4 litters, only two of which survived. Sequence analysis showed that one of the two litters contained a single gene-edited pup with a mutation in the Peromyscus Rosa26 locus.

While we successfully employed i-GONAD to engineer Peromyscus deletions, the method proved to be highly inefficient, yielding only one gene-edited pup after nearly 40 surgeries (FIG. 1B). This poor efficiency may be partially attributed to a low rate of fertilization among the females subjected to i-GONAD, since vaginal lavage was used to detect sperm, which is an unreliable pregnancy marker in Peromyscus leucopus [Dewsbury & Lanier, Physiology & Behavior, 17(6), 921-924 (1976)]. Moreover, pregnant Peromyscus that were repeatedly handled may have experienced pregnancy block, a phenomenon which has been reported to occur at a high rate in experimentally manipulated pregnant Peromyscus [Utt, College of William and Mary—Arts and Sciences (1984)]. Collectively, these efforts underscored the need for a more efficient approach to generating timed pregnant Peromyscus, which are required for nearly every transgenesis method.

Estrous Tracking with Core Body Temperature

Timed mating is required for a variety of transgenesis protocols, including the generation of 1) embryos at a precise gestational age, 2) recipient mice suitable for embryo transfer, or 3) to implement iPSCs/ESC-based transgenesis. In Mus, achieving efficient pregnancy requires either properly timing the estrous state of female mice with mating to ensure receptivity to male studs, and/or checking females for vaginal plugs to determine if mating has occurred [Byers, et al., PloS One, 7(4), e35538 (2012)]. The latter technique cannot be used with Peromyscus because their vaginal plugs are internal and undetectable [Hartung et al., Journal of Mammalogy, 59(4), 717-723 (1978)]. Researchers have previously employed vaginal cytology in Peromyscus to determine the stage of a mouse's estrous cycle, which is reported to span 4 to 5 days, but with high variability in the length (ranging from 2 to 8+ days) [Dewsbury et al., Journal of Mammalogy, 58(1), 89-92 (1977)]. When coupled with low timed pregnancy rates among P. leucopus subjected to vaginal lavage, existing estrous detection methods are demonstrably inadequate.

Over the course of the estrous cycle, mice experience numerous physiological changes, including changes in body temperature, which result from hormonal changes during the time of ovulation [Marrone, et al., Physiology & Behavior, 17(3), 419-425 (1976); Weinert et al., Biological Rhythm Research, 35(3), 171-185 (2004)]. In particular, the release of progesterone, which is critical for the establishment and maintenance of pregnancy, causes a corresponding rise in body temperature during estrus (FIG. 1C). As a result, core body temperature telemetry systems have been successfully employed in Mus musculus to resolve the estrous cycle for individual laboratory mice and arrange highly efficient timed matings [Smarr et al., PloS One, 11(7), e0160127 (2016)]. Core body temperature data collected by researchers using the aforementioned telemetry system supports a 4 to 5 day estrous cycle in Mus, as previously established [Byers, et al., PloS One, 7(4), e35538 (2012)]. Estrus can be clearly observed in mice with implanted temperature sensors as prolonged heat periods that extend into the daily rest period. We sought to apply the same telemetry system to Peromyscus to resolve the estrous cycle and precisely ascertain when individual mice are cycling. Briefly, sensors (G2 E-Mitters, Starr Life Sciences, Oakmont, PA) were implanted in the abdominal cavity of 10 to 16 week old female P. leucopus. These sensors track core body temperature and locomotor activity when coupled with a receiver that powers the sensor and collects measurement data over time (FIG. 1D). Temperature readings were collected continuously once per minute (FIG. 1E).

Following temperature sensor implantation, cycling patterns were observed among Peromyscus from the colony beginning 3 to 14 days after surgery. Without exception, a clear 4 day estrous cycle was observed in each cycling mouse, manifesting as a peak in core body temperature after the lights were turned on (FIG. 1F). This trend was apparent both for individual mice and for the population average (FIG. 1G). Contrary to previous findings, no variability in the length of estrous was observed; if a mouse began cycling, it cycled regularly every 4 days. In total, 57 temperature implant surgeries were performed and 36 cycling mice were identified. The rate of cycling across all experiments (63%) was consistent with the reported rate of cycling among P. leucopus in a previous study (61%) (FIG. 1H) [Dewsbury et al., Journal of Mammalogy, 58(1), 89-92 (1977)].

Pregnancy with Estrous Tracking

Studies were then preformed to establish whether mating Peromyscus in estrus, as determined by core body temperature, could boost the pregnancy rate from timed mating in this species. First, 15 female Peromyscus were monitored to determine the timing of their estrous cycles and then mated them with proven studs on the night of presumed estrus (FIG. 1I). After 4 days, suspended estrous cycles were observed in 14 out of 15 mice. The high rate of estrous cycle disruption is readily apparent on the population average (FIG. 1J). 11 of the 14 Peromyscus produced litters after 24 to 26 days, with an average litter size of 3.4 pups (FIG. 7). Importantly, when female mice were mated during diestrus, no disruption of the estrous cycle was observed (FIG. 1K) and periodic heat fluctuations continue unabated unlike estrus matings (FIG. 1L). Cycling disruption, therefore, appears to be indicative of pregnancy, which is contingent on timed mating in estrus.

Interestingly, the telemetry system also revealed a subset of mice that experienced pregnancy-like events; 3 mice exhibited a break in estrous cycling that lasted for at least 20 days. Such pregnancy-like events have been previously documented in Mus by researchers employing mouse telemetry to improve timed pregnancy [Smarr et al., PloS One, 11(7), e0160127 (2016]). However, without further study, we cannot be sure if the observed suspension of the estrous cycle is due to implantation failure, pseudopregnancy or miscarriage. Overall, mouse telemetry for estrous detection more than quadrupled the efficiency of pregnancy or pregnancy-like events in P. leucopus from 20% to 93% (FIG. 1M).

Estrous Tracking with Animal Activity

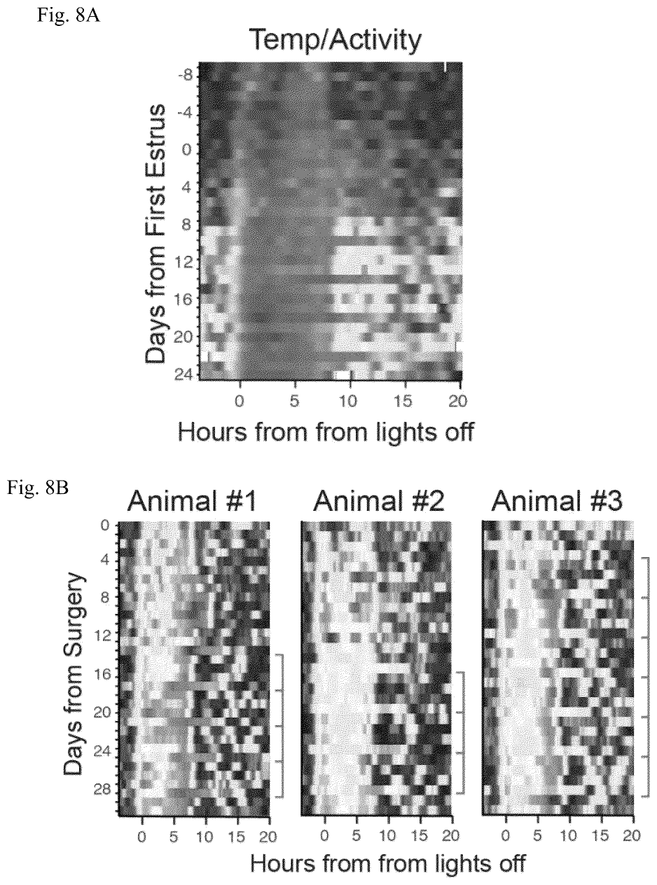

As with core body temperature, researchers have observed elevated activity on days of estrus [Smarr et al., PloS One, 11(7), e0160127 (2016)]. However, tracking mouse activity was previously deemed unreliable for estrous detection, since researchers could only distinguish estrus from averaged activity data, and not for individual mice [Smarr et al., PloS One, 11(7), e0160127 (2016)]. In light of these findings, initially studies were designed and carried out to focus efforts on resolving the Peromyscus estrous cycle from core body temperature readings alone, with a goal of ultimately detecting pregnancy or pregnancy-like events. However, once temperature-based estrous detection was established, it was observed that temperature readouts were highly correlated with concurrent activity data collected by the sensors (FIG. 8A). Notably, results permitted clear identification of a prolonged extension of activity coinciding with elevated temperature in individual mice on days of estrus (FIG. 8B), a trend that was similarly visible at the population level (FIG. 8C). Based on these findings, monitoring locomotor activity was determined to be equally effective for establishing estrous.

Although the mouse temperature-based telemetry system enabled efficient timed mating in Peromyscus, it was accompanied with significant drawbacks. First and foremost, each mouse was required to undergo an invasive sensor implantation surgery. Although surgery does not prevent cycling, it clearly disrupted the Peromyscus estrous cycle, as witnessed by the extended period between surgery and the first day of cycling, which spanned on average 13 days. Undoubtedly, the combined effects of post-surgical recovery and estrous disruption extend experimental timelines. Furthermore, mouse telemetry systems are expensive, with costs exceeding $1,575 per sensor set-up. This price point likely renders sensor-based estrous tracking infeasible for labs and transgenic cores to adopt at scale. As such, studies were performed to develop a new estrous tracking system based on activity detection that would reduce both the cost and experimental delays precluding the application of highly efficient timed matings to entire colonies.

### Development of Camera Tracking for Estrous Detection

Unlike mouse telemetry, camera technology is cheap, widely available, and noninvasive. Thus, studies were performed to develop a camera system for tracking Peromyscus activity using the Raspberry Pi, a tiny, single-board computer that has been widely adopted due to its low cost, modularity, and open design. Briefly, Peromyscus cages were equipped with a Raspberry Pi Zero 2 W (WiFi-enabled) computer attached to a Raspberry Pi NoIR V2 camera module capable of medium-resolution photographs (FIG. 2A). Given the small size of the camera set-up, three mouse racks were outfitted with 210 Raspberry Pi units to monitor the maximum number of mouse cages that can fit per rack (FIG. 2B). The camera module captured images at 4 second intervals, which were then processed on the Raspberry Pi units and the resultant activity data uploaded to our cloud database. Every two hours, this data was downloaded to the server, processed further, and cached for fast visualization. Every 6 hours, email notifications were sent out detailing any Pi units that hadn't successfully uploaded their data (FIG. 2C). A web interface was created with which to view activity patterns and simplify equipment monitoring and troubleshooting (FIG. 2D).

The G2 E-Mitter sensors detect movement through a voltage shift in an internal capacitor. Consequently, the act of scratching could register as activity as readily as lateral cage movements. Thus, initial studies were performed to establish if estrous cycling activity was detectable via camera by placing a Raspberry Pi camera adjacent to a cage with a mouse with an implanted sensor. In doing so, results indicated the system was able to simultaneously track both the core body temperature and locomotor activity data generated by the sensor, and any movement visible to the camera (FIG. 2E). Estrous cycling was easily identifiable by all three readouts, with estrus days as recognizable with the Raspberry Pi camera system as with telemetry. Additional observations indicated that the addition of a near-IR LED provided roughly comparable resolution during the daytime while also enabling night-time tracking (FIG. 2F). In contrast to the mouse telemetry system, the introduction of cameras permitted observation of cycling trends without the delays and discomfort caused by surgery. Furthermore, the cost of the camera setup is $75 per cage, which is less than 1/20 of the cost of mouse telemetry. Thus, the mouse camera system provided a cheap and effective alternative to invasive telemetry systems and was able to be readily implemented by any lab.

Insights from Camera Tracking

As the mouse colony grew, eventually more than a thousand female mice were placed under camera surveillance. The estrous cycling rate in the colony detected by camera mirrored the rate initially detected by mouse telemetry, confirming the effectiveness of the new approach (FIG. 2G). Female Peromyscus are believed to attain sexual maturity between 46 and 51 days of age [Clark, Journal of Mammalogy, 19(2), 230-234 (1938)]. Yet, monitoring a substantial cohort of prepubescent mice through sexual maturity, resulted in the identification of an overall later onset of estrus behavior (mean=11.5 weeks, sd=2.5 weeks) and a broader spectrum of potential estrus initiation ages (FIG. 2H). The first estrus behavior stands out distinctly from non-estrus activity when no discernible trends are present (FIG. 2I) and almost always exhibits a consistent 4-day cycle. Out of over 1000 female Peromyscus, fewer than 5 mice have been observed with 5-day cycles (FIG. 2J).

Historically, Peromyscus are not associated with sleep disorders or any irregularities in their sleep patterns. However, camera tracking also revealed that approximately 20% of the mice displayed shifted circadian rhythms (FIG. 2K). It was unclear whether this phenomenon was unique to the colony, a result of inbreeding, or indicative of a broader trend in the population. This altered sleep pattern became evident soon after mice were placed under camera surveillance and remained consistent; it neither developed gradually nor dissipated overtime. In some cases, estrous cycling could still be observed among mice with shifted circadian rhythms (FIG. 2L).

### Timed Mating for Embryo Production

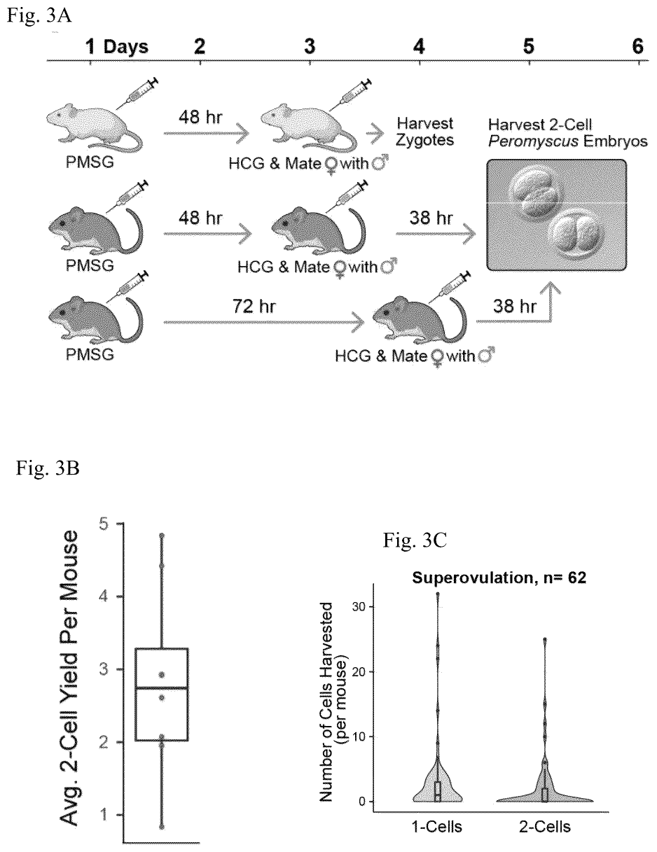

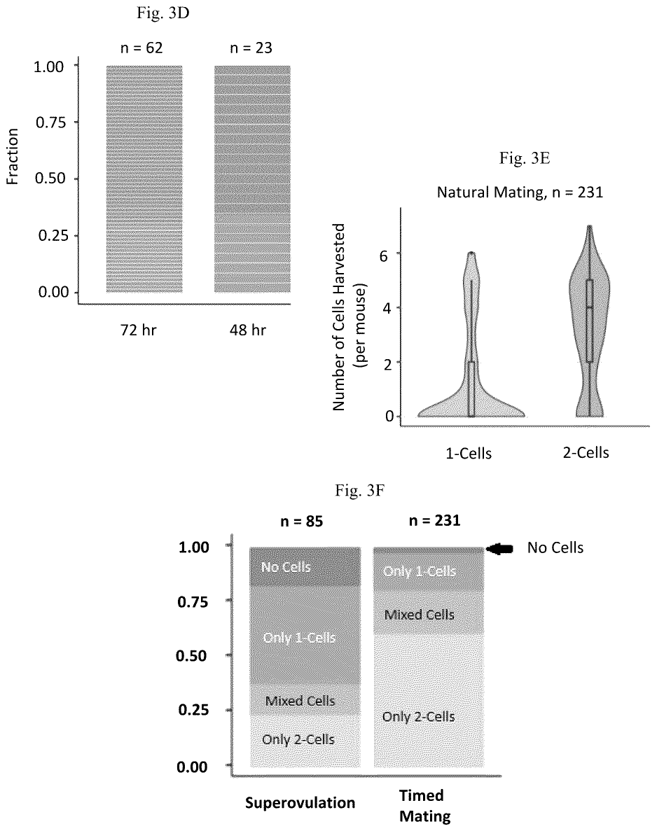

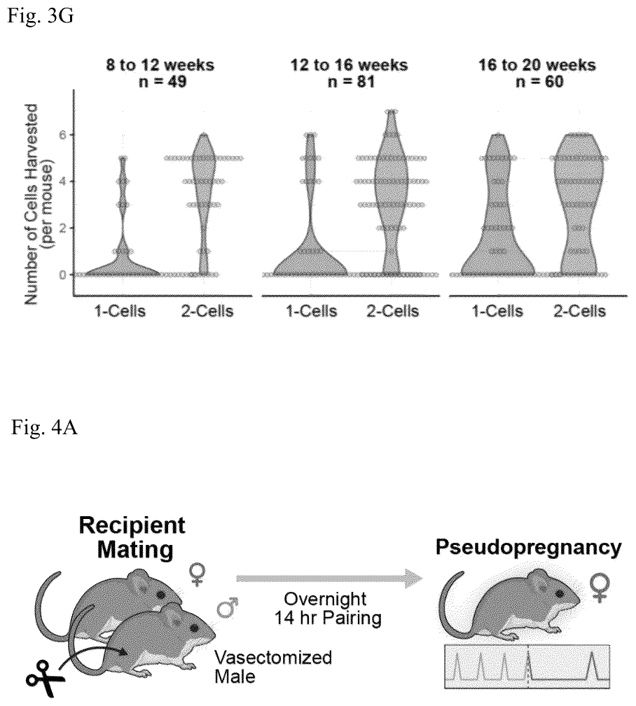

Having successfully implemented camera-based estrous tracking, studies were performed that employed timed mating at scale for embryo generation, a non-limiting example of which was to improve embryo generation for transgenesis. As previously mentioned, the superovulation protocol, which required a 72-hour gap between hormone administrations with embryos pooled, yielded poor results (FIG. 3A). Following superovulation, embryos from a group of mice were harvested and pooled. Despite many efforts, the average yield per mouse remained below five embryos, and displayed marked inconsistency, with one experiment yielding fewer than one embryo per mouse (FIG. 3B). To identify the source of variability, studies were carried out that included tracking the number of cells harvested from individual mice. It was quick determined that a small group of mice was producing the vast majority of cells (FIG. 3C), leading to experimental inconsistency. Additionally, our fertilization rate was far lower than anticipated; even if a mouse produced eggs after superovulation and mating, they were unlikely to be fertilized (FIG. 3D). Following the identification of a clear 4-day estrous cycle, we implemented the standard Mus superovulation regimen of 48 hours between hormone doses, but unfortunately observed equally low rates of fertilization (FIG. 3D).

Researchers have demonstrated that mouse oocytes and embryos produced without superovulation are of a superior quality to those produced after the exogenous administration of gonadotrophins [Ertzeid et al., Human Reproduction, 16(2), 221-225 (2001);(Lee et al., Molecules and Cells, 40(8), 558-566 (2017)]. Given the high pregnancy efficiency obtained with timed mating, coupled with the known embryo quality improvements, studies were then performed to compare timed mating and superovulation to assess embryo generation efficiency. Briefly, mice were tracked via camera monitoring, and then mated on the night of presumed estrus. Embryos were then harvested and counted per mouse. This approach yielded far more uniform results; the variation in cells per animal diminished greatly in comparison to superovulation (FIG. 3E), while the number of fertilized embryos per mouse surged (natural mating mode embryos/mouse=5, superovulation mode embryos/mouse=0). Timed mating was highly efficient at embryo generation; when mice were paired during estrus, they consistently generated eggs and the majority were fertilized (FIG. 3F). Younger timed-mated mice had greater embryo yields and fewer unfertilized eggs, whereas older mice displayed reduced productivity (FIG. 3G). Given the observed inconsistencies, lower yield, and fertilization rate with superovulation, coupled with the superior quality of embryos obtained through natural mating, the latter appeared to be a more reliable and effective method for embryo production in P. leucopus.

Pseudopregnancy Generation with Estrous Tracking

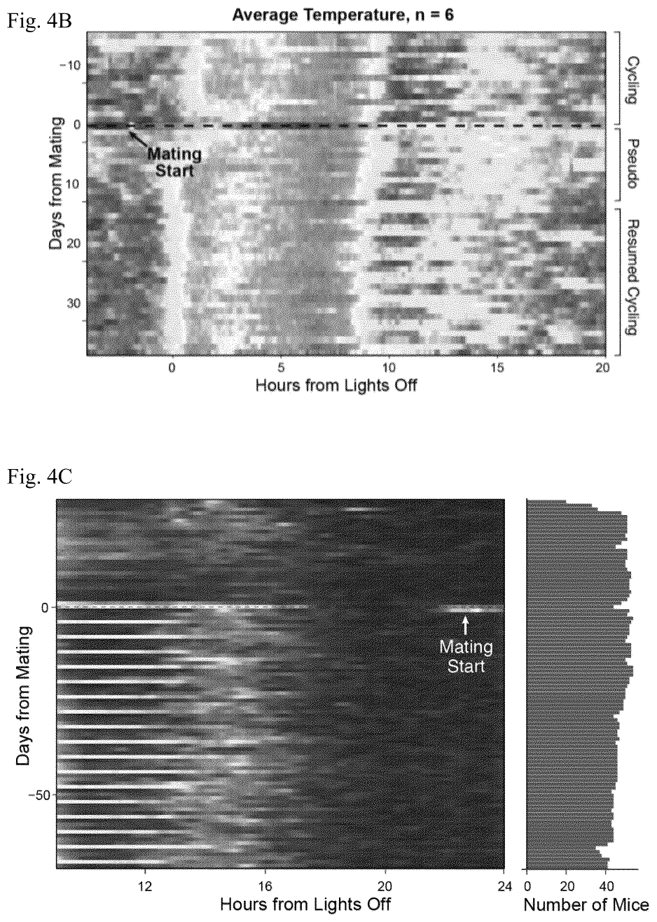

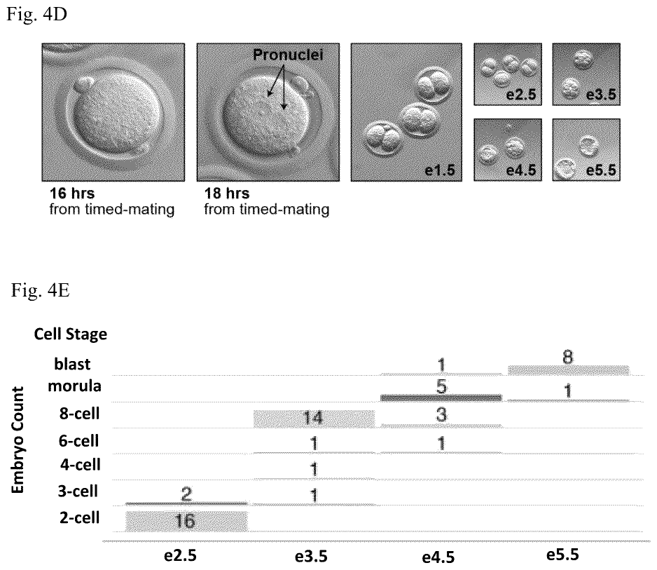

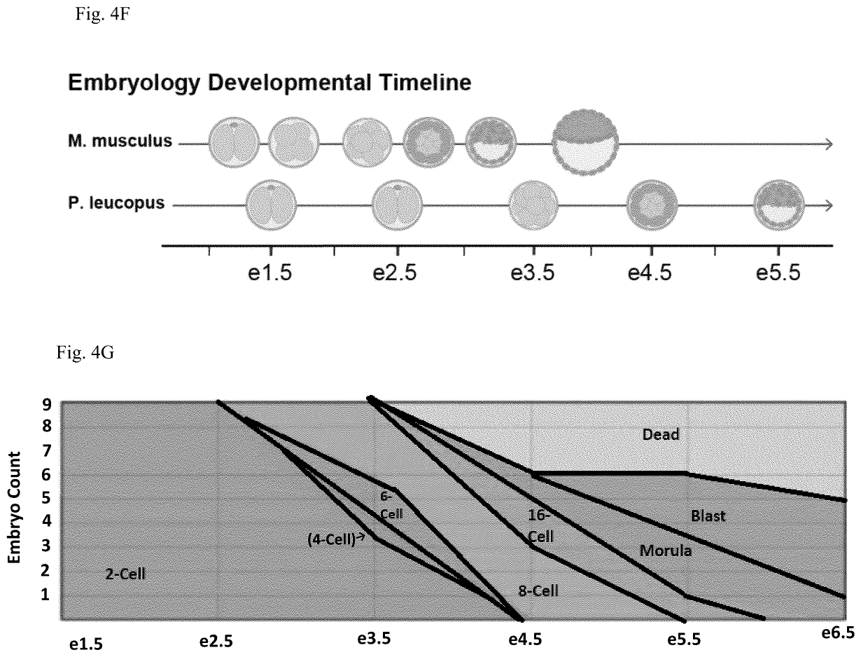

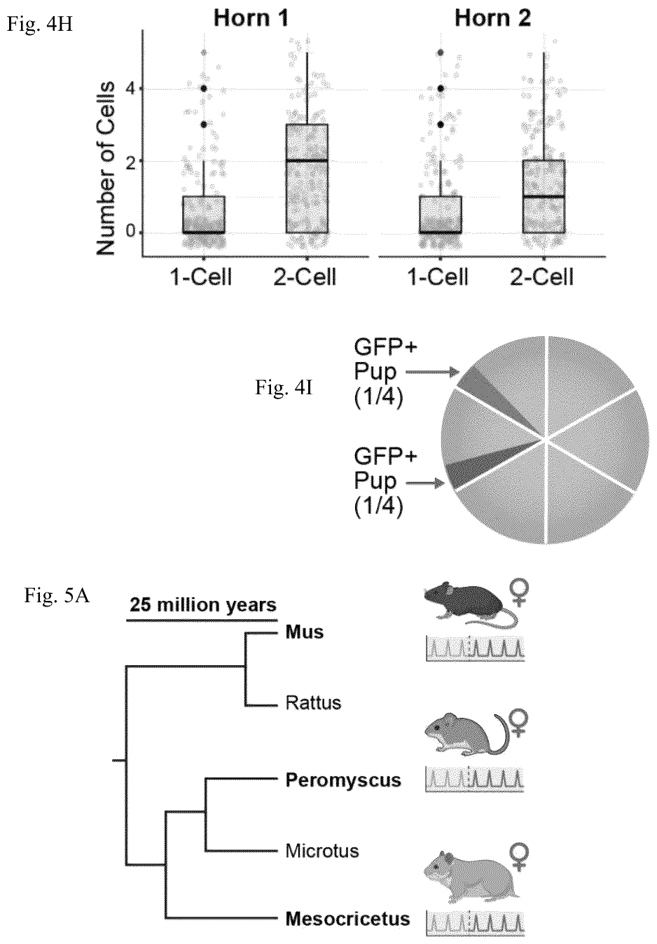

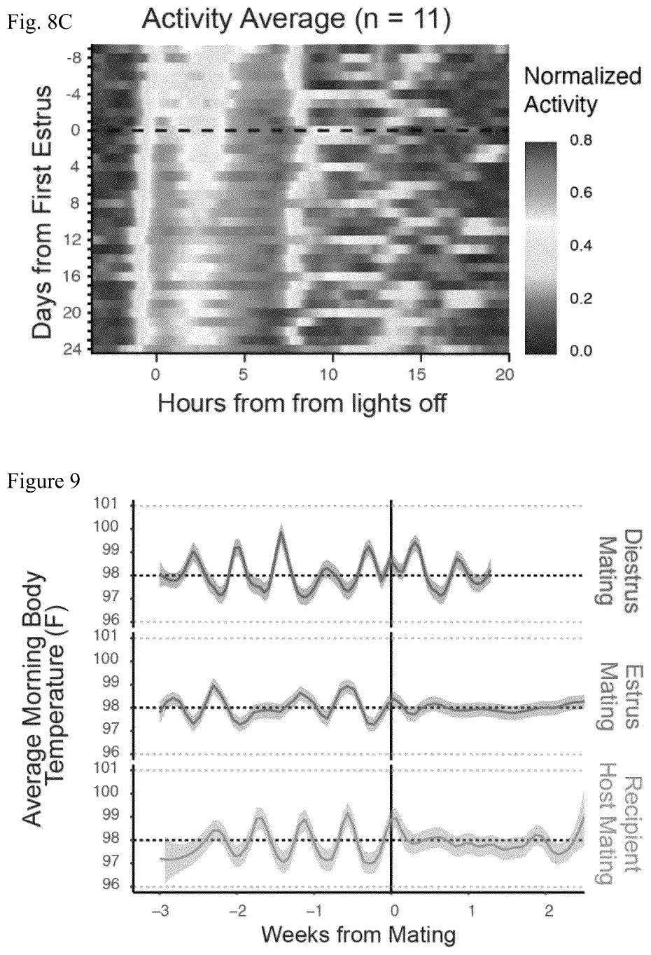

Most prior transgenesis protocols require the efficient generation of pseudopregnant recipients through the identification of copulatory plugs, which are not readily observable in P. leucopus. Experiments were performed using estrous tracking to enable the efficient generation of pseudopregnant mice. Briefly, male Peromyscus were vasectomized and sterility confirmed through mating experiments. Next, 6 female Peromyscus were tracked with implanted temperature sensors. Once estrous was established, females were mated on the night of presumed estrus with vasectomized males (FIG. 4A). After 4 days, suspended estrous cycles were observed in 5 of the 6 mice. The high rate of estrous cycle disruption is apparent among the population average (FIG. 4B). All mice resumed cycling roughly 2 weeks later (range=10 to 14 days). When core body temperatures are compared, pseudopregnancy closely resembles pregnancy in Peromyscus, except in length (FIG. 9). A total of 54 camera-tracked female Peromyscus were mated with vasectomized males and then subjected to various interventions for the experiments discussed in the subsequent sections. Of the 53 mice with data post mating, 85% ( 45/53) had suspended estrous cycling (FIG. 4C). Interestingly, for 6 of the 8 mice that continued to cycle, their cycle shifted by one day post mating, indicating that nearly all the mice mated, and that mating did not always invariably lead to pseudopregnancy. Similar cycle disruptions were observed, albeit at a lower rate, when swabbing Peromyscus for vaginal cytology.

P. Leucopus Developmental Timelines in vivo & in vitro Although the mid-to-late developmental timeline for P. maniculatus is well-documented, there is scarce information on the early developmental stages crucial for implementing embryology protocols in P. leucopus [Davis et al., PloS One, 11(3), e0150598 (2016)]. Further, the prior established timelines are impacted by inherent variability stemming from the lack of reliable timed matings. To establish a comprehensive developmental timeline for Peromyscus leucopus to determine the optimal timings for embryo harvest and transfer, forty timed matings were performed using camera-tracked female mice. Embryos were recovered for daily early gestational time points (FIG. 4D) and throughput development. Pronuclei became visible 13 hours post-mating (or after 18 hours in total), assuming fertilization occurs at the midpoint of the dark cycle. In contrast, the Mus pronucleus appears 5 to 11 hours after fertilization. For the early gestational stages, the cell types retrieved at each time point were mapped (FIG. 4E). Compared to Mus musculus, Peromyscus embryos exhibit extended two-cell stages, persisting until e2.5, and demonstrate delayed blastocyst development, with blastocysts forming on day e5.5 (FIG. 4F). Blastocyst implantation appeared to occur on or before e6.5 as no cells were found at this time point. The extended two-cell phase in Peromyscus could potentially increase the efficiency of genomic editing, given a potentially higher rate of homologous recombination during a longer G2 phase [Gu et al., Nature Biotechnology, 36(7), 632-637 (2018)].

Because transgenesis protocols often necessitate extended overnight embryo culture, studies were performed to characterize the in vitro development of Peromyscus embryos. Harvested 2-cell embryos from timed-mated mice were cultured in KSOM and housed in a tri-gas incubator until blastocysts formed. Embryos were imaged daily in order to construct an in vitro developmental timeline (FIG. 4G). It was observed that 44% of the embryos developed to the blastocyst stage, and that development was delayed in vitro, with blastocysts appearing on day e6.5. Given these findings, a decision was made to forgo the standard overnight culture protocol to avoid developmental delays, culture and instead harvesting was done at the 2-cell stage.

### P. Leucopus Embryo Transfer & Pronuclear Injection

In preparation for embryo transfer, studies were performed to identify the ideal number of embryos to transfer by studying the inherent capacity of Peromyscus oviducts. Camera-tracked female Peromyscus were mated on the night of presumed estrus, then harvested embryos on gestation day e1.5, recording quantities from each individual uterine horn (FIG. 4H). Noting a maximum of five embryos per uterine horn, it was determined that transferring four embryos would have the greatest potential for successful implantation.

Next unedited embryos were transferred into pseudopregnant Peromyscus to establish a baseline implantation rate, with the eventual goal of transferring engineered embryos. Briefly, two groups of female mice, embryo donors and embryo recipients were organized, and their estrous cycles were established using camera-tracking. To synchronize reproductive stages, donors were mated with proven studs a day before recipient matings with vasectomized males. This scheduling allowed for the harvest and implantation of two-cell embryos on the same day, eliminating the need for overnight incubation. Embryos were harvested on gestation day e1.5 in M2 and underwent brief culturing in KSOM within the tri-gas incubator. Finally, four two-cell embryos were transferred into one oviduct for each recipient. In total, 12 embryo transfers of unedited Peromyscus cells were performed. 6 recipients produced a total of 15 pups or midgestation fetuses, as the embryo transfer efficiency improved over time (from 2/6 to 4/6 recipients). These findings demonstrate the viability of embryo transfer in Peromyscus, thereby enabling rederivation.

With embryo transfer established, studies were then performed to demonstrate genome engineering by performing pronuclear injection with CRISPR/Cas9 using a targeting construct without a selection marker (pR26-CAGGS-EGFP). Two Peromyscus leucopus Rosa26 targeting construct design used in certain experiments are shown in Table 1.

TABLE 1
Rosa26 targeting construct designs.

1 kb Rosa HA - CMV Promoter - EGFP - Bgh-pA - 1 kb Rosa HA
1 kb Rosa HA - CAG Promoter - EGFP - T2A - Puro - Bgh-pA - 1 kb
Rosa HA

Camera-tracked donors were mated with proven studs one day prior to the mating of camera-tracked recipients with vasectomized males to synchronize reproductive stages. Two-cell embryos (e1.5) were harvested, and each blastomere was injected with the targeting construct, Cas9 and Rosa26 gRNA. A total of 24 CRISPR-injected 2-cell embryos were transferred into 6 recipients (4 per mouse). Six recipients received four CRISPR-injected embryos each; from this, two recipients each yielded a single GFP+ pup, resulting in two transgenic Peromyscus (FIG. 4I). The two delivered pups each were GFP-positive, resulting in an 8% conversion rate with 100% of delivered pups containing the insert (FIG. 4I). By comparison, previous attempts to engineer the Mus Rosa26 locus (by 2-cell injection with a comparable 4.5 kb insert), generated a total of 2 founders from 22 pups following transfer of 75 microinjected embryos, for a 3% conversion rate with only 9% of pups containing the insert [Gu et al., Nature Biotechnology, 36(7), 632-637 (2018)]. These advances demonstrate the practical application of CRISPR technology in generating transgenic Peromyscus, paving the way for genetic investigations in this species.

### Camera Tracking of Other Rodent Species

For over a century, researchers have attempted to link physical activity and estrous state in different species with mixed results [Slonaker, American Journal of Physiology-Legacy Content, 68(2), 294-315 (1924); Smarr et al., PloS One, 11(7), e0160127 (2016); Wollnik & Dohler, Physiology & Behavior, 38(2), 229-240 (1986); and Wollnik & Turek, Physiology & Behavior, 43(3), 389-396 (1988)].

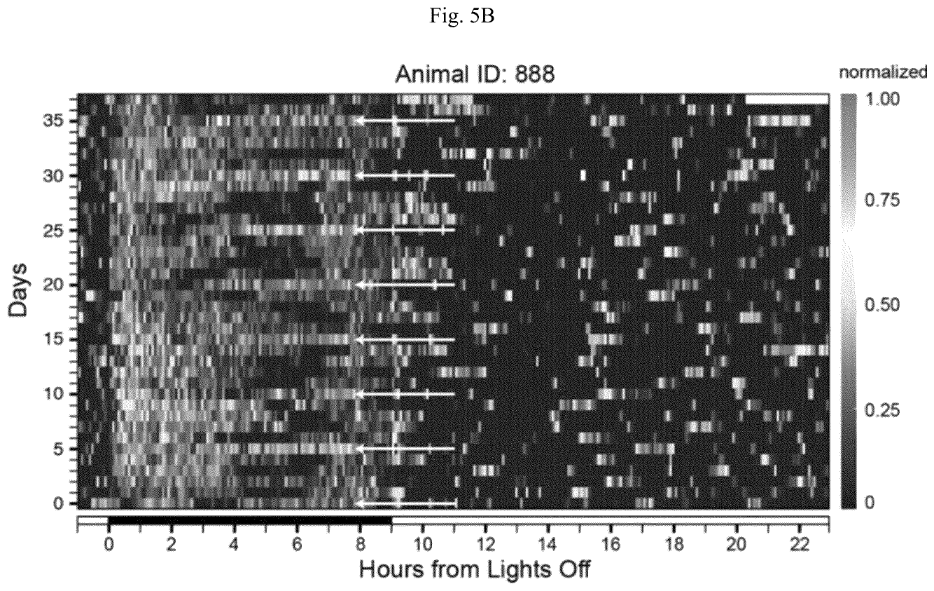

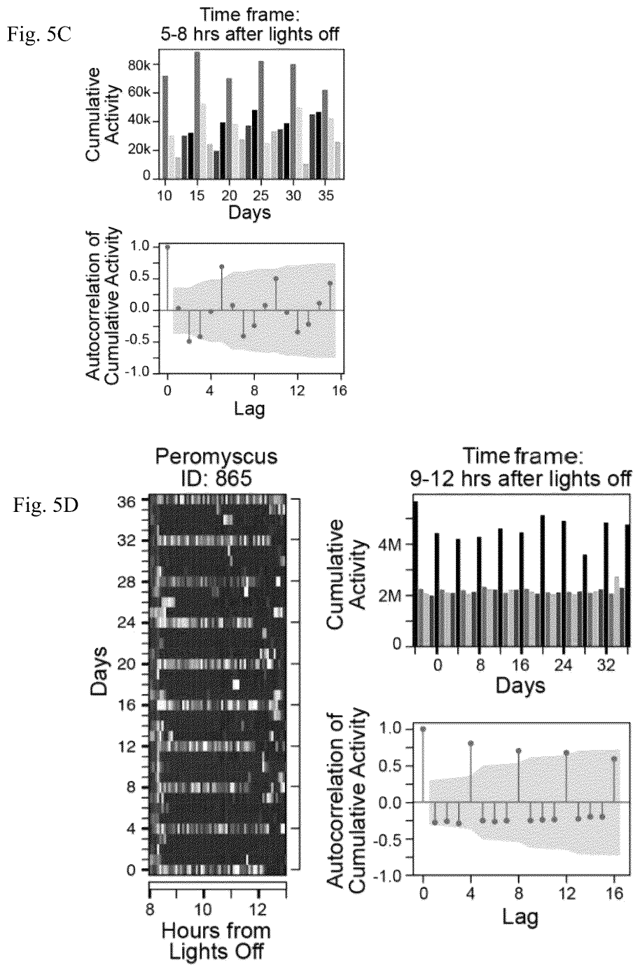

Studies were next performed to determine whether camera-tracking could be used to identify periodic, cycling activity in other rodent species. As proof of concept, the camera-tracking system was used to monitor female C57BL/6 Mus musculus, where estrous activity is reportedly undetectable at the individual level [Smarr et al., PloS One, 11(7), e0160127 (2016)], and Syrian hamsters Mesocricetus auratus, a species previously studied for activity patterns during their estrous cycles (FIG. 5A) [Prendergast et al., Journal of Biological Rhythms, 27(3), 246-256 (2012)]. For both Mus and hamsters, behaviors associated with estrous cycling were previously reported to manifest at night [Prendergast et al., Journal of Biological Rhythms, 27(3), 246-256 (2012); Smarr et al., PloS One, 11(7), e0160127 (2016)], in contrast to P. leucopus, which extends into the morning. Thus, having previously validated nighttime camera-tracking with additional IR illumination, IR light strips (940 nm) were used to illuminate the cages. IR cameras were used to track a total of 14 female Mus musculus and 5 female hamsters. Shortly after the animals were placed on camera, a 5-day cycling pattern between the hours of 6 and 9 UTC became apparent for one of the Mus (FIG. 5B). However, these periodic activity trends were much harder to discern by eye from the raw data for both Mus and hamsters.

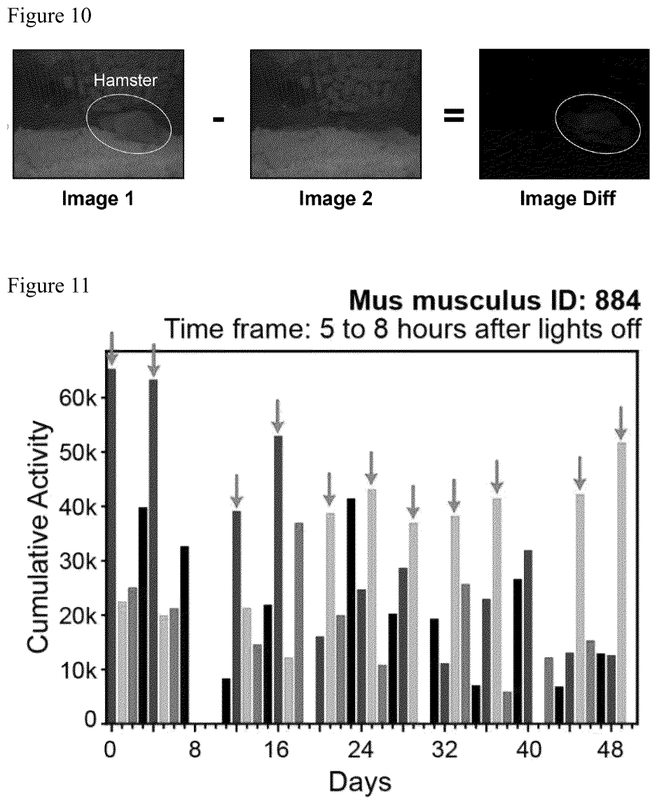

The camera tracking system detects activity via pixel-wise subtraction on two successive images (see methods). When images are taken under near infra-red illumination, several issues impede the detection of nocturnal animal activity. Under IR lighting, some animals become indistinguishable from the background (FIG. 10). Further, confounding artifacts such as reflections on the cage walls and wire, shifting bedding, flickering IR light and intrinsic sensor noise, may be misinterpreted as movement. To improve activity detection, background subtraction was applied to images using the “TwoPoint” model (see methods), which classified an image as either foreground or background. Additionally, each image was sectioned into quadrants, and the quadrant containing the food hamper was used exclusively for analysis. Following image processing, the one Mus with visible cyclical behavior showed periodic cumulative activity 5 to 8 hours after lights off, autocorrelation confirmed the significance of this observed 5-day trend (FIG. 5C).

A significant positive spike at lag 5 and a negative value at lag 2 suggests an inverse relationship within this 5-day cycle, indicating alternating high and low activity days. By comparison, Peromyscus with clear periodic behavior (9 and 12 hours after lights off) had much lower variability in daily cumulative activity and consequently longer periods of statistically significant autocorrelation (see lags 4, 8, and 12) (FIG. 5D). Using only autocorrelation, periodic cycling behaviors were observed among a larger group of Mus and hamsters. While image processing did not greatly improve visual trend detection in Mus, daily cumulative activity revealed periodic 4 or 5-day patterns 5 to 8 hours after lights off (FIG. 5E), with varying significance. Interestingly, one Mus displayed a shift in its cycling day over time, a behavioral change rarely seen in Peromyscus without mating (FIG. 11).

Following image processing, more hamsters also showed periodic 4-day daily cumulative activity patterns, with varying significance levels confirmed through autocorrelation. To further improve activity tracking, near-IR cameras were substituted with thermal cameras. Subsequently, hamster activity trends became visually apparent by activity graph 1 to 4.5 hours after lights off. While moving cage elements occasionally interfered with the signal, the periodicity of the 4-day cycling trend gained even greater statistical confidence (FIG. 5F). Collectively, these results suggest that simple, low-cost camera-tracking using IR or thermal cameras is a viable method for monitoring periodic behavior across a broad range of species, regardless of time of day or activity level.

### Improved Peromyscus Vaginal Cytology Using Camera-Based Activity Tracking and Machine Learning

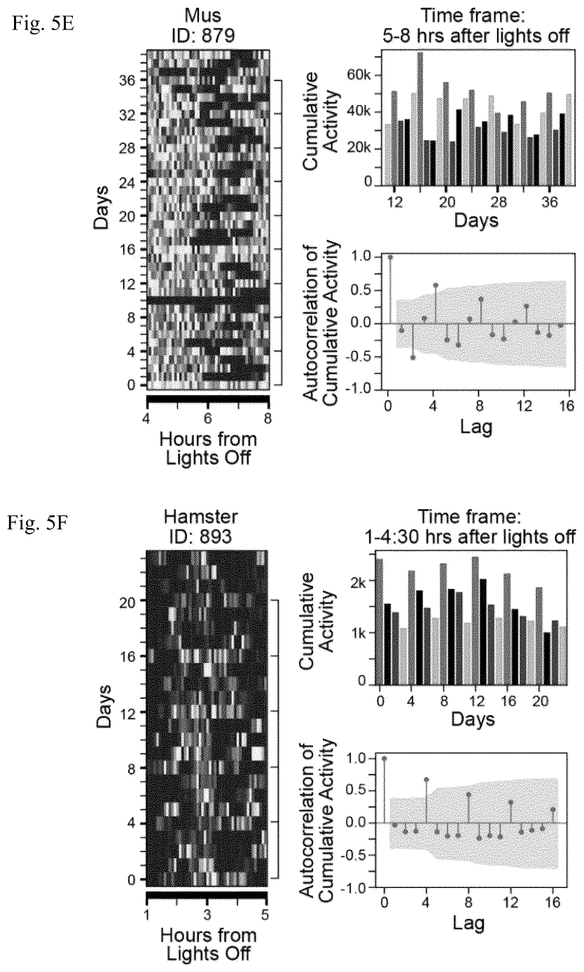

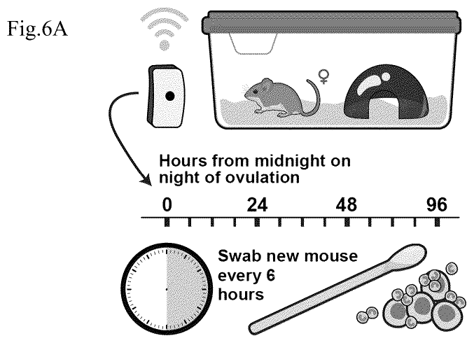

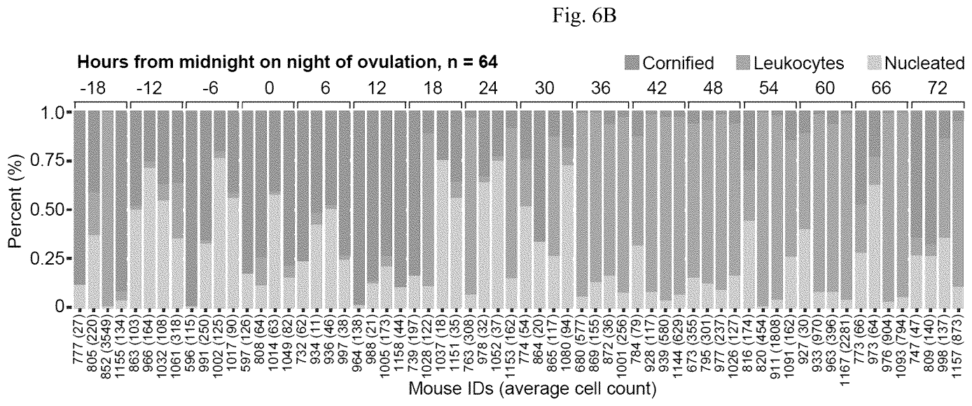

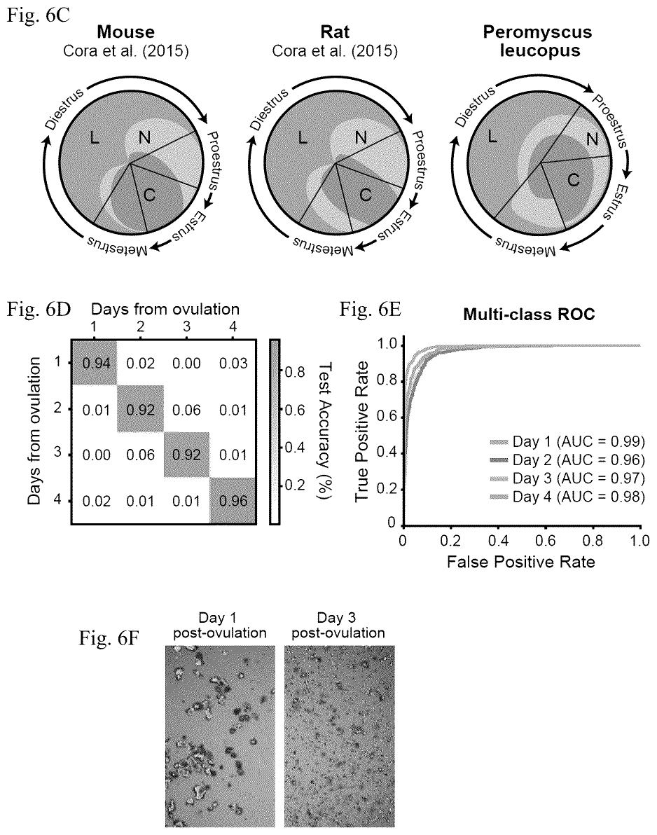

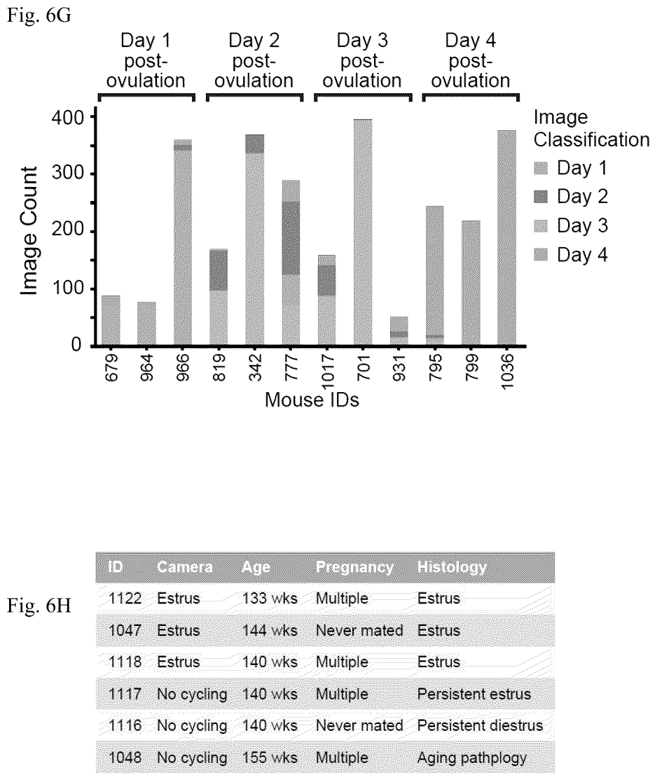

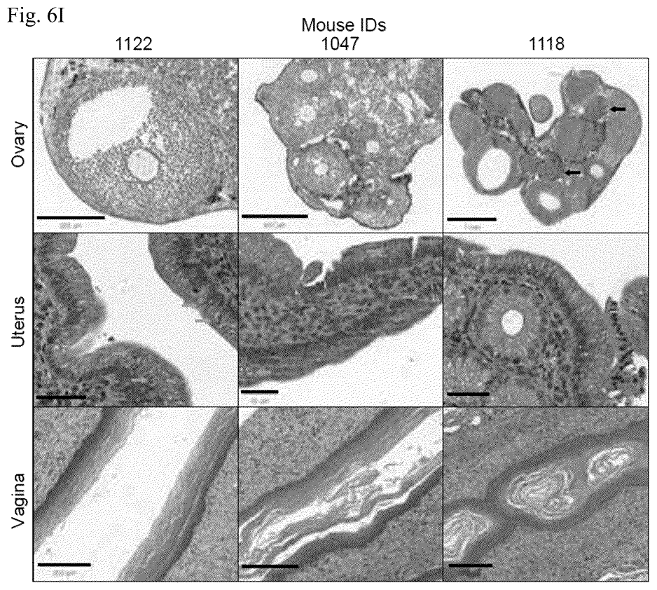

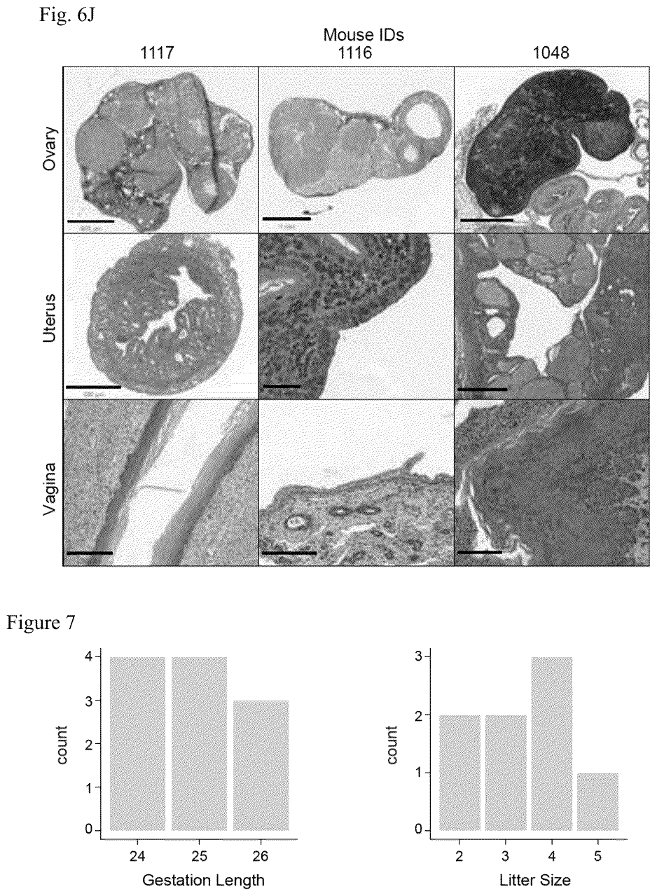

In contrast to previous reports of high variability using lavage and vaginal cytology in peromyscus, camera tracking revealed highly periodic estrous cycling behavior. It was postulated that camera tracking could improve vaginal cytology by reducing variability, because samples could now be collected from mice in the identical estrous stage. Ideally, cytology collected over the 4-day estrous cycle from camera-tracked mice would reveal distinct readouts for each stage, and thus, improve cytology for resource-limited labs without the space, time, or personnel to set up cameras. Briefly, vaginal samples were collected from groups of 4 Peromyscus every 6 hours from midnight on the night of ovulation with a total of 96 mice swabbed (FIG. 6A). Whole slides with vaginal samples were scanned, resulting in 100's of images per mouse. Next, 3 highly confluent images from each mouse were chosen and cells were counted, averaged, and plotted according to their collection time (FIG. 6B). Clear patterns emerged from the cytology data, including a long period predominated by leukocytes, in contrast to prior reports of a relatively short leukocyte-dominant period, or diestrus, and long estrus [Paull, J. A. & Fairbrother, J. Exp. Zool. 233, 143-149 (1985)]. Indeed, P. leucopus vaginal cytology appears to resemble Mus or rat cytology (FIG. 6C).

To demonstrate that camera-tracking had reduced variability among samples collected at the same estrous stage while improving differentiation of samples collected at different stages, a machine learning algorithm was trained to distinguish the 4 days of the P. leucopus estrous cycle.

Briefly, 120 different camera-tracked mice were swabbed at 9 am, either 9, 33, 57, or 81 hours from midnight on the night of ovulation. Whole slides were scanned and segmented images were screened for blurriness and cell density, with poor quality images removed. Finally, 12 k images were fed into the ResNet-50 model, which has been previously used for automated classification of Mus and rat cytology [Wolcott, N. S. et al. Sci. Rep. 12, 17685 (2022)]. The model was validated with 3 k images and showed classification accuracy for all four days of estrous (FIG. 6D) and clear separation among the days by their multi-class ROC curves (FIG. 6E). The research included studying how images were classified by overlaying gradient-weighted class activation maps to visualize the parts of the images that were most important for the model's decisions (FIG. 6F). Highlighted features had biological relevance, indicating that the model aligns with the cytology findings.

Finally, the model was tested on images from 12 new mice, not used for training or validation. Although the model associated 75% of mice with the correct estrous day, classification is more nuanced at the individual image level (FIG. 6G). Images from the first and fourth day post ovulation were consistently classified correctly, while the second day was often confused with the third. Therefore, staging by cytology with this protocol may require swabbing the same mice over successive days for confidence. However, we identified estrous cycle skipping or pseudopregnancy in 4% of swabbed mice, and the likelihood of cycle disruption increases with successive swabbing days.

### Camera-Based Activity Tracking for Monitoring Female Reproductive Aging

In this study, over 1,000 mice were monitored, inadvertently establishing a small cohort of old mice valuable for reproductive aging research. Six camera-tracked aged females-3 cycling and 3 non-cycling-were chosen for necropsy to examine differences in their female reproductive tracts (FIG. 6H). The 3 cycling mice, examined on their anticipated estrus day, exhibited typical estrus signs akin to rat estrus, with resemblances to mouse and hamster estrus. Ovaries had large antral F2 follicles and basophilic corpus lutea; uteruses featured a degenerating endometrial lining with tall columnar surface epithelium and luminal dilation; vaginas showed a shedding cornified layer, with sloughed cells and debris in the lumen, yet retained much attached cornified epithelium [Dixon, D. et al. J. Toxicol. Pathol. 27, 1S-107S (2014) and Westwood, F. R. Toxicol. Pathol. 36, 375-384 (2008)](FIG. 6I) (Refer to Methods for histological analysis).

All non-cycling mice displayed unique aging phenotypes previously undocumented in Peromyscus; one mouse exhibited characteristics of persistent estrus, including tall columnar endometrial epithelium, early endometrial hyperplasia, and vaginal epithelium cornification—traits linked to persistent estrus in mice/rats; another mouse presented signs of persistent diestrus, characterized by low, cuboidal, inactive endometrial epithelium and low, somewhat mucified vaginal epithelium, as anticipated in rats; the final mouse showed signs that it was nearing anestrus, with an atrophic ovary and endometrial cystic hyperplasia—a condition described in aging rodents which dysregulates the estrous cycle—and additional evidence of hormonal dysregulation/persistent estrus in the vagina Dixon, D. et al. J. Toxicol. Pathol. 27, 1S-107S (2014) and Westwood, F. R. Toxicol. Pathol. 36, 375-384 (2008)]. (FIG. 6J) (Refer to Methods for histological analysis). Thus, camera tracking methods provided a non-invasive strategy for identifying animals undergoing reproductive aging, thereby facilitating the study of the effects of hormonal dysregulation without animal sacrifice or intrusive techniques.

### DISCUSSION

Peromyscus have been proposed as a model organism for human disease, as they appear to more closely resemble humans than laboratory mice by hematological measurements, methylation profiles, disease susceptibility, genetic diversity, and lifespan (which exceeds Mus in captivity by about threefold) [Sun, Y., et al. International Journal of Experimental Pathology, 95(5), 342-350 (2014); Horvath, S., et al., GeroScience, 44(1), 447-461 (2022); and Havighorst et al., Seminars in Cell & Developmental Biology, 61, 150-155. 2017]. However, it is widely acknowledged that the use of Peromyscus as a disease model has been impeded by the dearth of genomic tools and technologies available for this species [Havighorst et al., Seminars in Cell & Developmental Biology, 61, 150-155. (2017)]. The challenges associated with engineering this species were and are formidable, as evidenced by the many issues we encountered when propagating and expanding ESCs, generating self-sustaining iPSCs, culturing Peromyscus embryos, hormone priming mice, and even simply maintaining a large enough colony to support our experiments without financial strain. Much work remains to be done in all these areas.

Nevertheless, the advancements described herein mark the arrival of Peromyscus as a tractable model organism. Results of studies such as those described herein, provide highly optimized reproductive protocols for the generation of timed-pregnant and pseudopregnant Peromyscus via low-cost camera tracking, which rival the efficiency of timed matings in laboratory mice. Such protocols completely eliminated the need for plug checking, vaginal cytology, and sperm detection, which are not only invasive, but known to cause pregnancy block at high rates in this species. Leveraging these protocols, it has now been shown that efficient embryo generation and transfer are achievable in P. leucopus, thereby unlocking the use of the most powerful transgenesis techniques, namely zygote and stem cell-based technologies, in this species. Finally, results of studies such as those described herein demonstrated efficient Peromyscus genome engineering with CRISPR/Cas9, paving the way for the development of Peromyscus as homologous, isomorphic, and predictive disease models, as well as their use for ecological engineering projects, such as Mice Against Ticks, a community-guided effort to engineer Lyme-resistant Peromyscus [Buchthal et al., Philosophical Transactions of the Royal Society of London. Series B, Biological Sciences, 374(1772), 20180105 (2019)].

For more than a century, researchers have explored the relationship between physical activity and estrous states in various species, with varying degrees of success. A pioneering study published in the early 20th century was among the first to establish a link between rodent activity and estrous stages [Slonaker, American Journal of Physiology-Legacy Content, 68(2), 294-315 (1924]). However, subsequent studies challenged this correlation in mice and rats, contending that activity levels were not indicative of estrous cycling even in animals with previously established estrous cycles [Smarr et al., PloS One, 11(7), e0160127 (2016); Wollnik & Dohler, Physiology & Behavior, 38(2), 229-240 (1986)]. Consequently, success in detecting estrous-linked activity appears to hinge on the monitoring technology applied.

Prior work has not utilized camera-tracking for estrous detection, though it has become an essential tool for the study of animal behavior, providing unparalleled insight into behavioral development [Dunn et al., Nature Methods, 18(5), 564-573 (2021)], social learning [Hong et al., Proceedings of the National Academy of Sciences of the United States of America, 112(38), E5351-E5360 (2015)], neuropsychiatric disorders [Woodard et al., eneuro Vol. 4, Issue 5, pp. ENEURO.0141-17.2017 (2017)] and memory by tracing the locomotor activity of laboratory animals[(Seibenhenner et al., Journal of Visualized Experiments: JoVE, 96, e52434 (2015)]. Studies and results presented herein in which camera-based tracking was successfully applied to a larger group of rodents has expanded the utility of camera tracking, by broadening the range of detectable movements and enabling monitoring regardless of the time of day. The identification of species-specific, periodic activity in three evolutionarily distinct rodents suggests that cycling behaviors are widespread and can be determined in additional types of rodents.

Despite prior reports, it has now been shown that P. leucopus have highly periodic cycling behavior with consistent cycle length. Whereas previous investigations of Peromyscus cycle length relied on human inspection of vaginal cytology readouts [Dewsbury et al., Journal of Mammalogy, 58(1), 89-92 (1977)], camera-based activity tracking establishes a ground truth based on ovulation time, enabling large-scale sampling from staggered groups of identically staged mice. Studies presented herein evidenced clear trends in cell populations over the course of the estrous cycle, while amassing a dataset that rivals the most extensive databases of Mus and rat cytology Wolcott, N. S. et al. Sci. Rep. 12, 17685 (2022]. Subsequently, a machine learning model was trained that accurately identified two of the four estrous days, though the remaining two days had reduced accuracy due to similarities among cell populations. While consistent daily swabbing for the two high accuracy days will eventually resolve an animal's cycle, the risk of estrous cycle disruption increases with time, rendering camera tracking the preferred method for staging.

Studies presented herein also demonstrated use of camera-based activity tracking to monitor a cohort of old female Peromyscus, revealing distinct activity patterns that correlated with reproductive health. Histological analysis exposed aging etiologies among non-cycling elderly mice that otherwise appeared physically identical (without histological inspection) to their cycling counterparts. Mice of the same age had markedly different reproductive aging profiles, suggesting that this technology may aid our understanding of individual variations in reproductive aging, while identifying new behavioral biomarkers for ovarian conditions and improving predictions of reproductive lifespan. When combined with Peromyscus transgenesis protocols and advanced techniques, such as scRNAseq, spatial transcriptomics, and proteomics, camera tracking can facilitate longitudinal studies to investigate the underlying mechanisms of reproductive health and longevity. Aging Peromyscus colonies can be examined using methods of the invention to reveal novel fundamental mechanisms governing diseases of aging by providing insights into age-related changes in gene expression, protein function, and cellular metabolism.

With over 56 recognized species, the Peromyscus genus is celebrated for its extensive diversity, and the tools described herein are liable to unlock genome engineering for all Peromyscus species. Further, these advances hold promise beyond Peromyscus, and may extend the existing set of model organisms to include other intractable rodents, with major opportunities in less-studied systems for modeling human disease. Possible examples with translational potential include, but are not limited to, refractory species of rats, prairie voles, guinea pigs, and naked mole rats. By comparing well-established Mus strains to new rodent models, it can be possible to use genetic engineering to dissect the underlying mechanisms that influence longevity Holzenberger, M., et al., Nature, 421(6919), 182-187 (2003) and Liang, H., et al., Experimental Gerontology, 38(11-12), 1353-1364 (2003)]; neurogenesis [Kim, E. B., et al., 479(7372), 223-227 (2011)]; social behavior patterns [McGraw, L. A., & Young, L. J. Trends in Neurosciences, 33(2), 103-109. (2010)]; degeneration [Eimer, W. A., & Vassar, R. Molecular Neurodegeneration, 8, 2 (2013)]; and susceptibility to disease [Tian, C., et al., Nature Communications, 8(1), 599 (2017)]. Collectively, the methods presented herein represent a transformative opportunity for transgenesis in Peromyscus and other species and broaden the use of genetic engineering technologies in recalcitrant species.

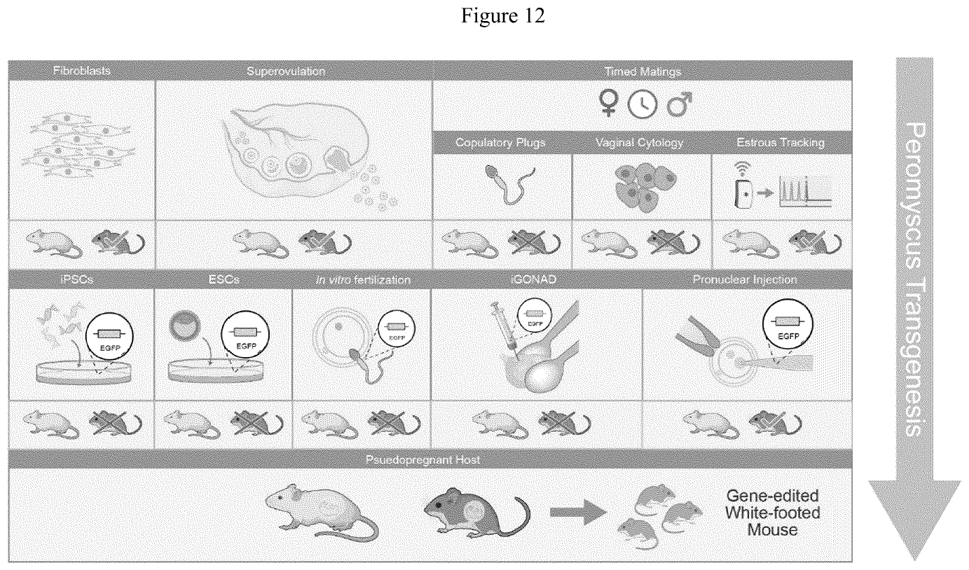

Finally, due to the abundance of wild P. leucopus in North America, and their role in the transmission of infectious disease, it is proposed to immunize local P. leucopus populations to be heritably resistant to B. burgdorferi, the etiological agent of Lyme disease [Telford et al., Lyme Disease and Relapsing Fever Spirochetes: Genomics, Molecular Biology, Host Interactions and Disease Pathogenesis (2021)]. In doing so, it is hoped to break the disease transmission cycle between wild mice and ticks, and to reduce disease prevalence in hard-hit areas like Nantucket and Martha's Vineyard [Buchthal et al., Philosophical Transactions of the Royal Society of London. Series B, Biological Sciences, 374(1772), 20180105 (2019)]. By combining high-efficiency estrous detection of the invention with CRISPR/Cas9 genome engineering, it is possible to generate wild P. leucopus that heritability express anti-Lyme antibodies derived from locally sourced mice [Schaible et al., Proceedings of the National Academy of Sciences of the United States of America, 87(10), 3768-3772 (1990)]. Engineered Peromyscus generated via camera-based estrous-tracking serves as a proof of concept for engineering rodents to prevent zoonoses ranging from Lassa fever to hantavirus pulmonary syndrome. FIG. 12 provides a schematic diagram of an example of Peromyscus transgenesis process.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention. All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements, and/or steps are in any way required for one or more embodiments or that one or more embodiments necessarily include logic for deciding, with or without other input or prompting, whether these features, elements, and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

All references, patents and patent applications and publications that are cited or referred to in this application are incorporated by reference in their entirety herein.

What is claimed is:

1. A method of identifying a reproductive state in a female mammalian test subject, the method comprising:
(a) measuring a level of movement activity of the female test subject in a first time period, wherein the movement activity is measured in the absence of a mating activity of the female test subject;
(b) comparing the level of movement activity of the female test subject measured in the first time period with a control level of movement activity; and
(c) determining a difference in the measured level of movement activity of the female test subject in the first time period compared to the control level of movement activity;
wherein the presence of the determined difference assists in identifying the reproductive state in the female test subject.

2. The method of claim 1, further comprising simultaneously measuring the level of movement activity in a plurality of female test subjects.

3. (canceled)

4. The method of claim 1, wherein the measuring comprises one or more of: detecting the movement activity with one or a plurality of sensors; visually detecting the movement activity; and imaging the movement activity with one or a plurality of cameras, optionally wherein one or more of the one or plurality of cameras is a thermal-imaging camera.

5-7. (canceled)

8. The method of claim 1, wherein the identified reproductive state includes at least one of proestrus, estrus, metestrus, diestrus, anestrus, ovulation, pseudopregnancy, and pregnancy.

9. The method of claim 1, further comprising (i) initiating a pregnancy in the female test subject when the identified reproductive state of the female test subject is estrus, optionally is ovulation, or (ii) initiating a pseudopregnancy in the female test subject when the identified state of the female test subject is estrus, optionally is ovulation.

10. (canceled)

11. The method of claim 1, further comprising (i) not initiating a pregnancy or pseudopregnancy in the female test subject when the identified state of the female test subject is not estrus, or (ii) not initiating a pregnancy or pseudopregnancy in the female test subject when the identified state of the female test subject is not ovulation.

12. (canceled)

13. The method of claim 1, further comprising initiating an environmental or physical change for the female test subject based on the reproductive state of the female test subject.

14-30. (canceled)

31. The method of claim 1, wherein the measurement comprises detecting the movement activity.

32. The method of claim 31, wherein the detecting comprises imaging the female test subject's movement activity with a camera or other visual recording device.

33. The method of claim 1, wherein the measuring of the movement activity of the female test subject comprises obtaining a plurality of images of the female test subject at preselected time intervals and comparing two or more of the obtained plurality of images to each other as a measure of movement activity of the female test subject.

34-35. (canceled)

36. The method of claim 33, further comprising performing pixel-level analysis of the two or more obtained images to detect the presence or absence of differences between the two or more images, wherein the detected differences identify movement activity by the female test subject.

37. The method of claim 36, further comprising identifying a level of movement activity of the female test subject in the first time period and in a second time period.

38. The method of claim 37, wherein identifying the level of movement activity of the female test subject in the first time period comprises comparing the pixel-level analysis of two or more of the images obtained in the first time period.

39. The method of claim 37, further comprising comparing the identified level of movement activity in the first time period to a level of activity identified in the second time period.

40-42. (canceled)

43. The method of claim 1, wherein the female test subject is a rodent, optionally a mouse or a hamster.

44-46. (canceled)

47. The method of claim 1, wherein the female test subject is a genetically engineered female test subject.

48. The method of claim 1 wherein the female test subject is a non-rodent mammal.

49-50. (canceled)

51. The method of claim 1, further comprising administering a candidate therapeutic agent to the female test subject, at least one or more of: before, during, and after the measuring.

52. (canceled)

53. The method of claim 1, wherein the female test subject is not a horse or a cow.

54. A computer-implemented method of identifying a reproductive state in a female mammalian test subject, the method comprising:
(a) receiving imaging data representing a plurality of images capturing movement activity of the female test subject, wherein the movement activity images are captured in the absence of a mating activity of the female test subject;
(b) measuring, using the imaging data, a level of movement activity of the female test subject in a first time period;
(c) comparing the level of movement activity measured in the first time period with a control level of movement activity; and
(d) determining a difference in the measured level of movement activity of the female test subject in the first time period compared to the control level of movement activity;
wherein the presence of the determined difference assists in identifying the reproductive state in the female test subject.

55-65. (canceled)


SEQUENCE LISTING (4 sequences)

SEQ ID NO: 1 - DNA, 17 bp, synthetic construct
  source 1..17: mol_type = other DNA
gggagttctctgctgcc

SEQ ID NO: 2 - DNA, 21 bp, synthetic construct
  source 1..21: mol_type = other DNA
agttttgctgcataaaacccc

SEQ ID NO: 3 - DNA, 1000 bp, synthetic construct
  source 1..1000: mol_type = other DNA
ggtggtcagggccatggtccgccctgccgcaaccggagggggtgggaggagggagcggaaagatctccaccggacgcggccatggctcccacggggggcggaggagcgcttccggttcccgtctcgtcgctgattggccttttctcctcccgccgtgtgtaaaaacacaaatggcgtgttttggttggagtgaagcgcctgtcagttaacgggtgctggagtgcgcagccgctggctgcctcgctgtgcccactgggtggggcgggaggtaggtggggtgaggagagctggacgtgcgggcgcggccggcccctggcggggcgggggaagggaggggagggagggtcagcgaaagtggctggcgcccaggcggcctcccaccctccccttcctctgggggagtcggtttacccgccgccggcctggcctcgtcgtctgattggctcccggggcccagaaaactggcccctgcaattggcccgcgttcgtgcaagttcagtccatacgcgggctggcgggggcggcagggaggcgctcgcaggttccggccctccccaaggccccgcgccgcagagtctggccccgcgcccctgcgcaacgtggcaggaagcgcgcgctgggggcggggacgggcaggcggtctgagcggcggggcgggggcgagcgcggttcctcctagagttgttgccgcacgaggggcgagctgaaccggacctgcatggcgcactcctaggagtggaggaaggagtgggggctcagtcgggccggtttggaggcaggaggcacttgctctccaaaattccgtgagagctgcaatcaataagggagccgcagtggagtaggcggggagaaggccgcacccttctcggcagggggaggggagtgccgcaatacctttctgggagttctctgctgcctcctggcttctaaagaccgccccgggcctggaacagtcccttccccctctcccctcgtgatctgcaagtcgaggctttctgga

SEQ ID NO: 4 - DNA, 1000 bp, synthetic construct
  source 1..1000: mol_type = other DNA
agatgggcgggagtcttctaggctggcttaagggctaacctggtgcgtgggcgttgtcctgcaggggaattgaacaggtttaaaattggagaaacgacacttcccacagattttcgtttgtgtccggagggaattgtaataggagcaaaggagggaaatgggtgactaggcgctcacctggggttttatgcagcaaaactacaggttattattgcttatgatctgccttgaagtatttttcatcgtgttagattaaagacatgctcacccaagtttatactcccacttgagatccttgttatatcatgaaattttagtatcgtaaattagaatatataaacagaatcttagcagtttctgcagagcccagtgcttcactctgtccctcttgctccctctgcagccctaccaagagatattttagcgctctctcattttagtcccgttttcatttgttgtactggcttatccaatccctagacagagcactggcattcttcctctcctgatcttagaagcctgatgagtcatgaaaccagacagattagttacaccacaaattgaggctgtagctagggccttgccctgcagttcttttattcctccttagtacattttgttgactgtttgccttgatttcattttctatccccacccttcaggagctttactacaatagatttattgagaatctaacctaaagttcagcttctttccactcccatgtatgcctatctcctttttcaccattaataaactgaactgttattaatggcttccagatggatgtctcctccaatatcacctgatgtatcttacatatcgtcaggctgattgtattaaaaggtacatgaattgacatgaaatttactgactccttaatgctttagtggattccatgagtacaatacagaagaccgttaatgggctactgacagctttttcatttcattatttcctgcatacacactttgagtcacatggaattgattactgctta